SEQUENCE LISTING (38 sequences)

SEQ ID NO: 1 - protein, 9 aa, Artificial Sequence
  Amino acid sequence derived from a survivin CTL epitope
  MISC_FEATURE (3)..(3): Xaa can be any naturally occurring amino acid
  MISC_FEATURE (5)..(5): Xaa can be any naturally occurring amino acid
  MISC_FEATURE (7)..(7): Xaa can be any naturally occurring amino acid
GWXPXDXPI

SEQ ID NO: 2 - protein, 9 aa, Artificial Sequence
  Amino acid sequence derived from a survivin CTL epitope
GWEPDDNPI

SEQ ID NO: 3 - protein, 9 aa, Artificial Sequence
  Amino Acid Sequence derived from a Mimotope
  MISC_FEATURE (4)..(4): Xaa can be any naturally occurring amino acid
  MISC_FEATURE (6)..(6): Xaa can be any naturally occurring amino acid
  MISC_FEATURE (8)..(8): Xaa can be any naturally occurring amino acid
AGPXAXAXL

SEQ ID NO: 4 - DNA, 57 bp, Artificial Sequence
  Nucleic acid primer sequence corresponding to a survivin CTL epitope
gtatattactgtgcgggttgggaaccagatgataatccaatatggggccagggaacc

SEQ ID NO: 5 - DNA, 57 bp, Artificial Sequence
  Nucleic acid primer sequence corresponding to a survivin CTL epitope
  misc_feature (22)..(23): n is a, c, g, or t
  misc_feature (24)..(24): k is c or g
  misc_feature (28)..(29): n is a, c, g, or t
  misc_feature (30)..(30): k is c or g
  misc_feature (34)..(35): n is a, c, g, or t
  misc_feature (36)..(36): k is c or g
gtatattactgtgcgggttggnnkccannkgatnnkccaatatggggccagggaacc

SEQ ID NO: 6 - DNA, 39 bp, Artificial Sequence
  Nucleic acid primer sequence corresponding to a survivin CTL epitope
tgatattcgtactcgagccatggtgtatattactgtgcg

SEQ ID NO: 7 - DNA, 40 bp, Artificial Sequence
  Nucleic acid primer sequence corresponding to a survivin CTL epitope
atgattgacaaagcttggatccctaggttccctggcccca

SEQ ID NO: 8 - protein, 9 aa, Artificial Sequence
  Variant of wild-type survivin epitope
GWFPLDAPI

SEQ ID NO: 9 - protein, 9 aa, Artificial Sequence
  Variant of wild-type survivin epitope
GWLPNDYPI

SEQ ID NO: 10 - protein, 9 aa, Artificial Sequence
  Variant of wild-type survivin epitope
GWRPTDVPI

SEQ ID NO: 11 - protein, 9 aa, Artificial Sequence
  Variant of wild-type survivin epitope
GWFPLDNPI

SEQ ID NO: 12 - protein, 9 aa, Artificial Sequence
  Variant of wild-type survivin epitope
GWIPSDFPI

SEQ ID NO: 13 - protein, 9 aa, Artificial Sequence
  Variant of wild-type survivin epitope
GWQPTDEPI

SEQ ID NO: 14 - protein, 9 aa, Artificial Sequence
  Variant of wild-type survivin epitope
GWTPKDDPI

SEQ ID NO: 15 - protein, 9 aa, Artificial Sequence
  Variant of wild-type survivin epitope
GWDPLDIPI

SEQ ID NO: 16 - protein, 9 aa, Artificial Sequence
  Variant of wild-type survivin epitope
GWQPMDSPI

SEQ ID NO: 17 - protein, 9 aa, Artificial Sequence
  Variant of wild-type survivin epitope
GWIPTDAPI

SEQ ID NO: 18 - protein, 9 aa, Artificial Sequence
  Variant of wild-type survivin epitope
GWCPYDTPI

SEQ ID NO: 19 - protein, 9 aa, Artificial Sequence
  Variant of wild-type survivin epitope
GWNPSDLPI

SEQ ID NO: 20 - protein, 9 aa, Artificial Sequence
  Variant of wild-type survivin epitope
GWVPTDLPI

SEQ ID NO: 21 - protein, 9 aa, Artificial Sequence
  Variant of wild-type survivin epitope
GWHPLDNPI

SEQ ID NO: 22 - protein, 9 aa, Artificial Sequence
  Variant of wild-type survivin epitope
GWNPFDGPI

SEQ ID NO: 23 - protein, 9 aa, Artificial Sequence
  Variant of wild-type survivin epitope
GWDPLDQPI

SEQ ID NO: 24 - protein, 9 aa, Artificial Sequence
  Variant of wild-type survivin epitope
GWAPNDNPI

SEQ ID NO: 25 - protein, 9 aa, Artificial Sequence
  Variant of wild-type survivin epitope
GWVPDDYPI

SEQ ID NO: 26 - protein, 9 aa, Artificial Sequence
  Variant of wild-type survivin epitope
GWQPVDRPI

SEQ ID NO: 27 - protein, 9 aa, Artificial Sequence
  Variant of wild-type survivin epitope
GWEPTDHPI

SEQ ID NO: 28 - protein, 9 aa, Artificial Sequence
  Variant of wild-type survivin epitope
GWCPQDLPI

SEQ ID NO: 29 - protein, 9 aa, Artificial Sequence
  Variant of wild-type survivin epitope
GWWPQDEPI

SEQ ID NO: 30 - protein, 9 aa, Artificial Sequence
  Variant of wild-type survivin epitope
GWFPLDVPI

SEQ ID NO: 31 - protein, 9 aa, Artificial Sequence
  Variant of wild-type survivin epitope
GWVPYDYPI

SEQ ID NO: 32 - protein, 9 aa, Artificial Sequence
  Variant of wild-type survivin epitope
GWRPVDPPI

SEQ ID NO: 33 - protein, 9 aa, Artificial Sequence
  Variant of wild-type survivin epitope
GWTPIDRPI

SEQ ID NO: 34 - protein, 9 aa, Artificial Sequence
  Mimotope Variant
  MISC_FEATURE (2)..(2): x is Glycine or Phenylalainine of
  MISC_FEATURE (4)..(8): x is anyaminio acid
  MISC_FEATURE (9)..(9): x is leucine or methionine
AXPXXXXXX

SEQ ID NO: 35 - protein, 9 aa, Artificial Sequence
  mimotope variant
AGPAAAAAL

SEQ ID NO: 36 - protein, 9 aa, Artificial Sequence
  Her2 derived epitope
TYLPTNASL

SEQ ID NO: 37 - protein, 9 aa, Artificial Sequence
  mouse Her2-derived epitope
TYLPANASL

SEQ ID NO: 38 - protein, 9 aa, Artificial Sequence
  Her2 epiotpe variant
  MISC_FEATURE (3)..(3): where x is any natural amino acid
  MISC_FEATURE (5)..(5): where x is any natural amino acid
  MISC_FEATURE (7)..(7): where x is any natural amino acid
TYXPXNXSL

(19) United States
(12) Patent Application Publication
(10) Pub. No.: US 2019/0339287 A1
(43) Pub. Date: Nov. 7, 2019

(54) IDENTIFICATION AND GENERATION OF PERSONALIZED VACCINE COMPONENTS BY FUNCTIONAL SCREENING VARIABLE EPITOPE AND MIMOTOPE LIBRARIES

(71) Applicant: Primex Clinical Laboratories, Van Nuys, CA (US)

(72) Inventor: Karen Manucharyan, Jardines En La Montana (MX)

(21) Appl. No.: 16/353,758

(22) Filed: Mar. 14, 2019

Related U.S. Application Data
(63) Continuation of application No. PCT/US17/51845, filed on Sep. 15, 2017.
(60) Provisional application No. 62/395,067, filed on Sep. 15, 2016.

(51) Int. Cl. G01N 33/68; G01N 33/50; C12N 15/10; A61K 39/00; A61P 35/00

(52) U.S. Cl. CPC G01N 33/6878; G01N 33/5011; C12N 15/1037; A61K 2039/812; A61P 35/00; G01N 2333/70539; A61K 2039/572; A61K 39/001106

(57) ABSTRACT

Specifically, methods for patient stratification and selection of personalized peptide based treatments and vaccines are disclosed, including assays for identifying antigenic and immunogenic peptides involved in immune responses of mammals against pathogens, cancer and other diseases by interrogating the T lymphocyte repertoire of a patient using combinatorial T-cell epitope and mimotope libraries.

### CROSS REFERENCE

This application is a continuation of International Application No. PCT/US2017/051845 filed Sep. 15, 2017, which claims priority to U.S. Provisional Application No. 62/395,067 filed Sep. 15, 2016, the contents of which are incorporated herewith in their entirety.

### BACKGROUND

It is clear that every individual is unique in appearance, behavior and genetic makeup. Frank, R. C., WEB MD; Dec. 1, 2011, teaches for example that for any specific stage and type of cancer, no two individuals can experience the disease in exactly the same way because their bodies and minds are unique. So it makes sense that both disease and medication affect us in unique ways.

One obstacle in treating cancer is its genetic variability which develops over time in an individual as a result of mutagenesis. Manucharyan, Karen et al. (US20020160199471) address this obstacle for

treating cancer and other diseases with antigenic variability through the use of variable epitope libraries (VELs) containing mutated versions of epitopes derived from antigens associated with the respective disease of interest. Manucharyan et al. teach compositions and methods encompassing the use of VELs targeting variable pathogens and disease antigens for treating subjects in both therapeutic and prophylactic settings.

While Manucharyan et al. addresses the treatment of disease involving antigenic variability, there is also a need in the art of patient treatment to address patient variability in responding to various therapeutics for diseases, including but not restricted to, diseases having antigenic variability.

In the field of cancer epitope vaccines, the modified, optimized or variant peptides, also known as altered peptide ligands (APLs), mimotopes, heterocyclic peptides or peptide analogues, bearing mutated versions of natural epitopes derived from tumor associated antigens (TAAs) considered as promising candidates for the development of vaccines [Platsoucas C. D. et al., (2003) Anticancer Res. 23(3A):196996; Jordan K. R. et al., (2010) Proc Natl Acad Sci U S A., 9; 107(10):4652-7; Hoppes R. et al., (2014) J Immunol. 193(10):4803-13]. Comprehensive screening strategies, such as screening a combinatorial

peptide library or testing virtually every single amino acid substitution within an epitope by a genetic screen, may lead to identification of superagonist APLs capable of eliciting potent antitumor patient-specific CTL responses where the native tumor-associated epitope fails [Abdul-Alim C S et al. (2010) J Immunol., 1; 184(11):6514-21; Ekeruche-Makinde et al., (2010) J Biol Chem. 2012 Oct. 26; 287(44):3726981]. The therapeutic agents/vaccines that incorporate peptide mimics of TAAs, or mimotopes, are thought to function by eliciting increased numbers of T cells that cross-react with the native tumor antigen, although often these T cells
have low affinity for the native tumor antigen [Buhrman et al., (2013) J Biol Chem. 2013 Nov. 15; 288(46):33213-25]. Interestingly, priming T cells with the mimotope, followed by a native tumor antigen boost, resulted in expansion of mimotope-elicited tumor-specific T cells with increased avidity for the native tumor antigen and improved antitumor immunity [Buhrman et al., (2013) Cancer Res.; 73(1):74-85].

### SUMMARY OF THE INVENTION

Methods are described herein for generating personalized combinatorial vaccines, the methods including the steps of identifying variants of specific epitopes of disease associated antigens and/or mimotopes of said epitopes and mimotopes variants thereof which are reactive with an individual's unique T cell repertoire, and tailoring a vaccine for the individual based on the responsiveness of the individual's immune system to the epitope(s), mimotope(s) and variants thereof. The personalized vaccines and related methods disclosed herein, which encompass some or all of the identified variants of the epitopes and/or mimotopes, can then be used in treatment of a disease or disorder, either alone or in combination with additional therapies. The personalized combinatorial vaccines generated have the potential of increasing the effectiveness of immunotherapy among the broader population.

Disclosed herein are the methods of the invention, including:
Methods to identify in each individual patient in advance of treatment, the character of the individual patient's specific T-cell responses with respect to relevant T cell epitope sequences and/or mimotope sequences and related sequences thereof.
Methods to customize combinatorial peptide vaccines for each individual patient based on the patient's T cell response results of the above.

The methods disclosed herein are useful in cancer therapies as well as to therapies and prophylaxis for infectious and other diseases.

### BRIEF DESCRIPTION OF THE DRAWINGS

FIGS. 1A-1XX is a table displaying 1000 randomly selected peptides from tumor antigen survivin epitope-derived VEL library bearing 8000 individual members.

FIG. 2 displays the results of PBMC cell proliferation from a patient afflicted with breast cancer against a panel of HER2 CTL epitope-derived VEL library mutant/variant epitopes VEL generated based on HER2-derived WT epitope sequence TYLPANASL (SEQ ID NO: 37), bearing the structural composition TYXPXNXSL (SEQ ID NO: 38), where the “X” is X is any amino acid. The data displayed in FIG. 2 represents the absolute numbers of % of proliferation. A non-related phage always resulted in low level background proliferation as did the majority of variant epitopes (data not shown).

### DETAILED DESCRIPTION

Personalized vaccines disclosed herein evaluate the interaction or recognition between receptors on the surface of an individual's T cells and a cell surface complex comprising an epitope and a Major histocompatibility protein (MHC). In developing personalized vaccines, a number of factors are considered, including the MHC alleles of an individual, the peptide epitopes generated by the individual, and the T cell repertoire displayed by the individual.

### MHC Class I and Class II Polymorphisms

As is well known in the art, there are two different classes of MHC molecules known as MHC class I and MHC class II, which deliver peptides from different cellular compartments to the surface of the infected cell. Peptides from the cytosol are bound to MHC class I molecules which are expressed on the majority of nucleated cells and are recognized by CD8+ T cells. MHC class II molecules, in contrast, traffic to lysosomes for sampling endocytosed protein antigens which are presented to the CD4+ T cells (Bryant and Ploegh, Curr Opin Immunol 2004; 16:96-102).

Also well known in the art is that peptide epitopes ranging from about 8-11 amino acids bind MHC class I molecules, while large peptide epitopes bind MHC Class II molecules Claus Lundegaard et al. “Major histocompatibility complex class I binding predictions as a tool in epitope discovery” Immunology. 2010 July; 130(3): 309-318. Human MHCs molecules, otherwise known as Human leukocyte antigens (HLA), are highly polymorphic (>2300 human MHC class I molecules encoding HLA-A and -B alleles have been registered by hla.alleles.org (http://hla.alleles.org/nomenclature/stats.html) and most of the polymorphisms influence the peptide binding specificity. As a result of this specificity

for peptide displayed by individual alleles of MHC molecules, a specified peptide epitope may bind a MHC Class I molecule of a first individual but not bind a MHC Class I molecule of a second individual. However, MHC alleles can be clustered into supertypes because many allelic molecules have overlapping peptide specificities which are not always obvious from the sequence similarity, as some alleles with very similar HLA sequences will have different binding motifs and vice versa.

### Generation of Peptide Epitopes

As is taught in the art, proteins expressed within a cell, including proteins (antigens) from intracellular pathogens or tumor associated antigens, are degraded in the cytosol by a protease complex, the proteasome, which digests polypeptides into smaller peptides, Claus Lundegaard et al., ibid. The protease is a multi-subunit particle, the beta ring of which contains three active sites, each of which is formed by a different subunit: B1, B2 and B5, each of which has different specificities, cleaving preferentially on the carboxylic side of either hydrophobic residues (B5), basic residues (B1), or acidic ones (B2), respectively. In certain cells, or in the presence of gamma interferon, these subunits may be replaced by an alternate set of active site subunits (B1i/LMP2, B2i/MECL1, B5i/LMP7) which results in the production of a different set of peptides, For a review see Rock et al “Proteases in MHC class I presentation and cross-presentation” J. Immunol. 2010 Jan. 1; 184(1): 9-15. Thus the set of proteasome cleaved peptides generated by a cell varies depending on the cell type and/or its environment.

As is taught in the art, a subset of the proteasome-cleaved peptides is bound by the transporter associated with antigen presentation (TAP), Claus Lundegaard et al., ibid, for example. These TAP associated peptides are translocated into the endoplasmic reticulum where, depending on their length and amino acid sequence, they bind MHC class I molecules and are exported as a peptide: MHC class I complex to the cell surface. Thus, the surface of an individual's cells displays a unique distribution of peptide: MHC class I complexes. The cell surface peptide: MHC class I complex is available for recognition by a T cell receptor from the individual's repertoire of T cell receptors displayed on the surface of Cytotoxic T lymphocytes (CTLs).

### CTL Recognition of Peptides Associated with MHC Class I

As is taught in the art, Cytotoxic T lymphocytes (CTL)s detect infected or transformed cells by means T cell receptors on the surface of CD8+ T cells which recognize peptide epitopes bound and presented by one of three pairs of cell surface MHC class I molecules (e.g., human HLA-A, HLA-B, and HLA-C molecules). Recognition of a specified peptide epitope depends on many factors, including the ability of the peptide epitope to bind an individual's MHC class I molecule as discussed above, and the presence in an individual's T cell repertoire of CD8+ T cells having a cell surface T cell receptor able to recognize and interact with the cell surface [peptide epitope: MHC class I] complex. It is estimated that for an effective immune response, at least one T cell in a few thousand must respond to a foreign epitope, Mason D. (1998) Immunol Today 19:395-404.

### T Cell Repertoires Differ Among Individuals

The TCR repertoire of each individual is distinct from that of other individuals as a result of both genetic differences and TCR dependent differences in processing of TCR bearing T-cells.

As is taught in the art', the antigen recognition portion of the T cell receptor (TCR) has two polypeptide chains, a and 13, of roughly equal length. Both chains consist of a variable (V) and a constant (C) region. The V regions of each pair of chains of a TCR interact with the MHC-peptide complex. Each TCR V region is encoded by one of several V region gene segments (more than 70 human TCR Va genes and more than 50 human V(3 gene segments) which has rearranged with a Ja gene segment to encode the TCR a chain, and both a D and a 0.113 gene segment to encode the TCR [3 chain, see McMahan R H, et al. J Clin Invest. 2006; 116:2543-2551; Wooldridge L, et al., J Biol Chem. 2011; 287:1168-1177; Parkhurst M R, et al. J. Immunol., 1996; 157:2539-2548; Borbulevych O. Y., et al. J. Immunol. 2005; 174:4812-4820; Zaremba S., et al., Cancer Res. 1997; 57:4570-4577; Salazar E, et al., Int. J Cancer. 2000; 85:829-838, http://www.ncbi.nlm.nih.gov/pmc/articles/PMC2913210/. The TCR Va and TCR VI3 gene segments display considerable polymorphism, with many being situated in coding/regulatory regions of functional TCR genes and several causing null and nonfunctional mutations, Gras et al. J. Exp. Med. Vol. 207 No. 7 1555-1567.

Thus at least one component of the uniqueness of an individual's T cell repertoire is thought to originate at a genetic level, due to at least in part to any of the polymorphism of T cell receptor loci, the imprecise rearrangement of V region gene segments and N and P region addition.

As is taught in the art, clonal selection of lymphocytes expressing T cell receptors with particular antigenic specificities further individualizes a person's T cell repertoire, Birnbaum M E., et al., (2014) Cell. 2014 May 22; 157(5):1073-87; Hoppes et al., (2014) J Immunol. 193(10):4803-13; Abdul-Alim C. S. et al., (2010) J Immunol., 1; 184(11):6514-21; Ekeruche-Makinde et al. (2010) J Biol Chem. 2012 Oct.

### 26; 287(44):37269-81; Buhrman et al., (2013) J Biol Chem. 2013 Nov. 15; 288(46):33213-25; Kappler J. W. et al. (1987) Cell 49: 273-80; Hengartner H. et al.,. (1988) Nature 336: 388-90; Pircher H. et al., (1991) Nature 351: 482-5.

Though not bound by theory, clonal selection is thought to further selectively refines an already unique set of T cells based on affinity to self-proteins, the self-proteins containing multiple polymorphisms between individuals. The combination of T cell receptor variability at the genomic level, and subsequent clonal selection of the T cells based on the expressed T-cell receptor, and environmental influences thereon, are thought to contribute in providing a T-cell repertoire with a range of binding specificities that is unique to each individual.

It is estimated in the art that a single T cell receptor can recognize more than a million peptides, giving rise to significant T-cell cross reactivity, Wooldridge L, et al. J Biol Chem. 2011; 287:1168-1177, which can be exploited to augment (or antagonize) immune responses using mimotopes. Epitope variants contain amino acid substitutions in the peptide sequence of an epitope that can improve peptide binding affinity for the MHC (Parkhurst M. R., et al. J Immunol. 1996; 157:2539-2548; Borbulevych O Y, et al. J Immunol. 2005; 174:4812-4820) and/or alter the interaction of the [peptide-MHC Class I] complex, (Jonathan D. Buhrman and Jill E. Slansky, Immunol Res. 2013 March; 55(0): 34-47; McMahan R H, et al. J Clin Invest. 2006; 116:2543-2551; Zaremba S, et al. Cancer Res. 1997; 57:4570-4577; Salazar E, et al. Int J Cancer. 2000; 85:829-838). Mimotopes bind to the same TCR receptor as epitope, but are not derived from same antigenic AA sequence. Similarly, mimotope variants contain amino acid substitutions in the peptide sequence of a mimotope that can improve peptide binding affinity for the MHC and/or alter the interaction of the [peptide-MHC Class I] complex. Vaccination with mimotopes can be more immunogenic than native tumor antigens, enhancing tumor specific T cell expansion and functional recognition of tumor cells. Lundegaard et al., Immunology. 2010 July; 130(3): 309-318.

Thus, identifying which set of peptides comprising epitopes/mimotopes and variants thereof, are able to bind the specific cell surface MHC class I molecules of a given individual and subsequently interact with the unique repertoire of CTLs present in the given individual at a given time is critical in developing personalized vaccines and/or individualized immunotherapy directed against intracellular antigens such as those generated by infectious disease or cancer.

In one embodiment methods are disclosed herein which identify peptides comprising CD8+ T-cell epitopes and/or mimotopes and/or variants thereof, from combinatorial epitope and/or mimotope libraries, using screening assays based on in vitro lymphoproliferation of CD8+ T-cells. Additionally or alternatively, combinatorial peptide libraries comprising a collection of mimotope variants are generated. From these libraries, sets of randomly selected individual peptides are obtained, preferably using chemical synthesis. These peptides are then applied to various assays to test the ability of the peptides to induce proliferation of peripheral blood mononuclear cells of individual hosts. Conventional assays utilized to detect T cell responses include proliferation assays well known in the art including, but not limited to, lymphokine secretion assays, direct cytotoxicity assays, and limiting dilution assays, for example.

In one embodiment, methods are disclosed herein which identify a set of peptides for treatment against a disease or condition afflicting an individual, wherein the subset of peptides comprises (i) a T cell epitope of an antigen expressed in said individual and/or (ii) variants of said T-cell epitope, comprising:

(a) generating a combinatorial variable epitope library (VEL) wherein said VEL comprises a plurality of peptides, each said peptide comprising a T cell epitope or variant thereof, wherein the length of each said T cell epitope or variant thereof, ranges from 8 to 11 amino acids, wherein the amino acid residues at MHC class I-anchor positions of said T cell epitope and its variant are identical, wherein the sequence of said T cell epitope and said variant thereof differ in at least two residues,
(b)
(i) incubating said T cell epitope or a variant thereof, with peripheral blood mononuclear cells (PBMCs) from a healthy individual (or a population of healthy individuals) under conditions suitable for inducing proliferation of PBMCs;
(ii) incubating said T cell epitope or variant thereof, with PBMCs from said individual afflicted with said disease or condition under conditions suitable for inducing proliferation of PBMCs, wherein said afflicted individual has a MHC Class I haplotype which is similar to the MHC Class I haplotype of said healthy individual,
(iii) comparing the proliferation of said T cell epitope and of each said variant thereof, in step (b)(i) versus step (b)(ii), thereby identifying three peptide groups:
   (a) Group I—peptides which induce proliferation of PBMCs of said afflicted individual and in said healthy population;
   (b) Group II—peptides which induce proliferation of PBMCs of said afflicted individual but not in said healthy population; and
   (c) Group III—peptides which do not induce proliferation of PBMCs of said afflicted individual but induce proliferation in said healthy population

wherein each said peptide Group, or a combination of two or more of Groups I, II and III, identifies a set of peptides for treatment against said disease or condition afflicting said individual.

In another embodiment, methods are disclosed herein which identify a set of peptides for treatment against a disease or condition afflicting an individual, wherein the set of peptides comprises (i) a mimotope of a T cell epitope of antigen expressed in said patient and/or (ii) variants of said T cell mimotope, comprising:

(a) generating a combinatorial variable epitope library (VEL) wherein said VEL comprises a plurality of peptides, each said peptide comprising a T cell mimotope or variant thereof, wherein the length of each said T cell mimotope or variant thereof ranges from 8 to 11 amino acids, wherein the amino acid residues at MHC class I-anchor positions of said T cell mimotope and its variant thereof are identical, wherein the sequence of said T cell mimotope and said variant thereof differ in at least two residues,
(b)
(i) incubating said T cell mimotope or variant thereof, with peripheral blood mononuclear cells (PBMCs) from a healthy individual (or population of healthy individuals) under conditions suitable for proliferation of PBMCs,
(ii) incubating said T cell mimotope or variant thereof, with PBMCs from said individual afflicted with said disease or condition under conditions suitable for proliferation of PBMCs, wherein said afflicted individual has MHC Class I haplotype which is similar to the MHC Class I haplotype of said heathy individual (or population of healthy individuals);
(iii) comparing the proliferation of said T cell mimotope and of each said variant thereof, in step (b)(i) versus step (b)(ii), thereby identifying three peptide groups:
   (a) Group I—peptides which induce proliferation of PBMCs of said afflicted individual and in said healthy population;
   (b) Group II—peptides which induce proliferation of PBMCs of said afflicted individual but not in said healthy population; and
   (c) Group III—peptides which do not induce proliferation of PBMCs of said afflicted individual but induce proliferation in said healthy population;

wherein each said peptide Group, or a combination of two or more of Groups I, II and/or III identifies a set of peptides for treatment against said disease or condition afflicting said individual.

In one embodiment, epitopes/mimotopes and variants thereof bearing “absolute immunogenicity” are a first vaccine/therapeutic agent component candidates. The “absolute immunogenicity” is defined as those epitopes/mimotopes and variants thereof showing the highest capacity to induce the proliferation of PBMCs obtained from the afflicted individual relative to PBMCs obtained from healthy subjects.

In another embodiment, epitopes/mimotopes and variants thereof showing decreased level of cell proliferation in the afflicted individual compared to cells from healthy subjects are component candidates for a second vaccine/therapeutic agent.

In another embodiment, epitopes/mimotopes and variants thereof, showing similar immunogenicity using the cells both from healthy individuals and patients with cells are a third vaccine/therapeutic agent component candidates.

In one embodiment, the potency of these groups as a component in a therapeutic agent or a vaccine can be determined using animal and/or preclinical models.

In one embodiment, the combinatorial epitope and/or mimotope peptide libraries comprise fusion proteins, each fusion protein comprising a peptide epitope, or a variant of the peptide epitope, or a peptide mimotope, or a variant of the peptide mimotope, enabling the selection of peptides capable of inducing proliferation of peripheral blood mononuclear cells of individual hosts.

The epitope or mimotope is preferably mutated to produce libraries, including combinatorial libraries, preferably by random, semi-random or, in particular, by site-directed random mutagenesis methods, preferably to exchange residues other than the Anchor positions of the MHC Class I T cell epitope. Anchor positions are very restricted in the choice of amino acids and are typically located at residues #2 and 3, near N-terminal end, and positions #8, 9, 10 or 11, near COOH-terminal end of a MHC Class I T cell peptide epitope or mimotope, or variant thereof.

Preferably, the combinatorial library is a “Variable epitope library” (VEL) that generates peptides reactive with an individual's repertoire of T cell receptors that target antigens expressed as a result of infectious disease or an internal disease or disorder, e.g., cancer. In one embodiment the target antigens are variable expressed in the host individual. In another embodiment, the target antigens are expressed as altered antigens due to mutagenesis or genetic instability. In one embodiment, a VEL library contains mutated variants of a CTL epitope, preferably a dominant CTL epitope, where 30-50% of amino acids at positions within the epitope other than the anchor positions are replaced by one of the 20 natural amino acids or derivatives thereof. In another embodiment, a VEL library contains mutated variants of a CTL mimotope, preferably a mimotope of a dominant CTL epitope, where 30-50% of amino acids at positions within the epitope other than the anchor positions are replaced by one of the 20 natural amino acids or derivatives thereof. Any of the known mutagenesis methods may be employed to generate the epitope variants and the mimotope variants, including cassette mutagenesis. These methods may be used to make amino acid modifications at desired positions of the peptide epitope or mimotope. In one example, VEL compositions disclosed herein may be prepared by expression in a bacterial, viral, phage display, or eukaryotic expression system. In another example, the VEL compositions may be expressed and displayed on the surface of a recombinant bacteriophage, bacterium or yeast cell. The complexity of the library or vaccine composition can be up to about 20⁸ synthetic peptides.

A preferred method according to the invention refers to a randomly modified nucleic acid molecule coding for an epitope or mimotope, or a variant thereof which comprises at least one nucleotide repeating unit within non anchor positions having the sequence 5′-NNN-3′, 5′-NNS-3′, 5′-NNN-3′, 5′-NNB-3′ or 5′-NNK-3′. In some embodiments the modified nucleic acid comprises nucleotide codons selected from the group of TMT, WMT, BMT, RMC, RMG, MRT, SRC, KMT, RST, YMT, MKC, RSA, RRC, NNK, NNN, NNS or any combination thereof (the coding is according to IUPAC).

The term “antigen” encompasses molecules or structures known to interact or capable of interacting with a T Cell Receptor (TCR) and/or a B cell receptor (BCR).

Substructures of antigens are generally referred to as “epitopes” (e.g. B-cell epitopes, T-cell epitopes), as long as they are immunologically relevant, i.e. are also recognizable by antibodies and/or T cell receptors. T cell epitopes are generally linear epitopes of antigens and can be classified based on their binding affinity for mouse major histocompatibility complex (MHC) alleles. MHC class I T cell epitopes are generally about 9 amino acids long, ranging from 8-10 amino acids, while MHC class II T cell epitope are generally longer (about 15 amino acids long) and have less size constraints.

As is well-known in the art, there are a variety of screening technologies that may be used for the identification and isolation of desired peptide proteins capable of associating with MHC molecules, to form a complex recognized by a T cell receptor, with certain binding characteristics and affinities, including, for example, display technologies such as phage display, ribosome display, cell surface display, and the like, as described below. Methods for production and screening of variants are well-known in the art.

Peripheral blood mononuclear cells (PBMCs) can be used as the source of CTL precursors. Those peptides able to induce in vitro proliferation of host peripheral blood mononuclear cells identify epitopes and/or mimotopes and/or variants thereof, to serve as a molecular component of personalized vaccines against cancer, infectious agents or other diseases in an individual host both in prophylactic and therapeutic settings.

Antigen presenting cells are incubated with peptide, after which the peptide-loaded antigen-presenting cells are then incubated with the responder cell population under optimized culture conditions. Positive CTL activation can be determined by assaying the culture for the presence of CTLs that lyse radio-labeled target cells, either specific peptide-pulsed targets or target cells that express endogenously processed antigen from which the specific peptide was derived. Alternatively, the presence of epitope-specific CTLs can be determined interferon secretion assays or ELISPOT assays, including Interferon gamma (IFNy) in situ ELISA.

In accordance with these embodiments, the composition of an epitope of a pathogen-specific nucleic acid or polypeptide disclosed herein may be selected from one or more epitopes of viral pathogens, e.g., Human Immunodeficiency Virus (HIV), Simian Immunodeficiency Virus (SIV), Hepatitis A, Hepatitis B, Hepatitis C, rhinovirus, influenza virus, plasmodium falciparum, tuberculosis, in addition to cancer related antigens, e.g., one or more epitopes of a tumor associated antigen (TAA).

Tumor associated antigens, include, but are not limited to, EpCAM, tumor-associated glycoprotein-72 (TAG-72), tumor-associated antigen CA 125, Prostate specific membrane antigen (PSMA), High molecular weight melanoma-associated antigen (HMW-MAA), tumor-associated antigen expressing Lewis Y related carbohydrate, Carcinoembryonic antigen (CEA), CEACAM5, HMFG PEM, mucin MUC1, MUC18 and cytokeratin tumor-associated antigen.

Also included are bacterial antigens, viral antigens, allergens and allergy related molecules. Additional antigens include, but are not limited to those of human cytomegalovirus (HCMV) gH envelope glycoprotein, HIV gp120, HCMV, respiratory syncytial virus RSV F, Hepatitis B gp120, Cytomegalovirus (CMV), HIV IIIB gp120 V3 loop, respiratory syncytial virus (RSV) Fgp, Herpes simplex virus (HSV) gD glycoprotein, HSV gB glycoprotein, HCMV gB envelope glycoprotein, Clostridium perfringens toxin and fragments thereof

Substructures of antigens are generally referred to as “epitopes” (e.g. B-cell epitopes, T-cell epitopes), as long as they are immunologically relevant, i.e. are also recognizable by antibodies and/or T cell

receptors. A T-cell epitope is the collective features of a peptide fragment, such as primary, secondary and tertiary peptide structure, and charge, that together form a site recognized by a T cell receptor or MHC/HLA molecule. Alternatively, an epitope can be defined as a set of amino acid residues necessary for recognition by T cell receptor proteins and/or Major Histocompatibility Complex (MHC) receptors. Epitopes are present in nature, and can be isolated, purified or otherwise prepared or derived by humans. For example, epitopes can be prepared by isolation from a natural source, or they can be synthesized in accordance with standard protocols in the art. Variants of synthetic epitopes can comprise artificial amino acid residues, such as D isomers of naturally-occurring L amino acid residues or non-naturally-occurring amino acid residues such as cyclohexylalanine. Throughout this disclosure, epitopes may be referred to in some cases as peptides or peptide epitopes.

T cell epitopes are generally linear epitopes of antigens and can be classified based on their binding affinity for mouse major histocompatibility complex (MHC) alleles. MHC class I T cell epitopes are generally about 9 amino acids long, ranging from 8-12 amino acids, while MHC class II T cell epitope are generally longer (about 15-22 amino acids long) and have less size constraints.

T cell epitopes of antigens associated with a particular disease or condition, such as tumor associated antigens (TAAs) associated with cancer, can be preliminarily identified using prediction tools known in the art, such as those located at the Immune Epitope Database and Analysis Resource (IEDB-AR), a database of experimentally characterized immune epitopes (B and T cell epitopes) for humans, nonhuman primates, rodents, and other animal species (http://tools.immuneepitope.org/analyze/html/mhc_binding.html).

Programs are available which provide high-accuracy predictions for peptide binding to human leucocyte antigen (HLA) -A or -B molecule with known protein sequence, as well as to MHC molecules from several non-human primates, mouse strains and other mammals). Lundegaard et al., Immunology 2010 July; 130(3): 309-318.

Mimotopes are peptides mimicking epitopes, preferably mimicking MHC class I binding epitopes. Mimotopes represent a close approximation of the original 3D-epitope, even though their amino acid composition rarely shows similarities. This is due to the fact that mimotopes mimic an epitope by their biochemical and electrostatic properties, and not necessarily by sequence homology. Thus, the term “mimotope” as used herein refers to any amino acid sequence that comprises substantially similar homology and/or biological activity as a wild type amino acid sequence. Similar homology may be determined by amino acid sequence identity and/or physico-chemical similarity. Similar biological activity may be determined by similarity in secondary, tertiary, and/or quaternary structure between the wild type sequence and the peptide mimotope.

With the phage display technology it is possible to generate such structural mimics of T-cell epitopes.

“T cell Repertoire”, on a nuclear level means a set of distinct recombined nucleotide sequences that encode T cell receptors (TCRs), or fragments thereof, in a population of T-lymphocytes of an individual, wherein the nucleotide sequences of the set have a one-to-one correspondence with distinct T-lymphocytes or their clonal subpopulations for substantially all of the T-lymphocytes of the population. In one aspect, a population of lymphocytes from which a repertoire is determined is taken from one or more tissue samples, such as peripheral blood monocytes (PBMC)s.

VEL libraries and VEL vaccine compositions disclosed herein can be administered to a subject prophylactically or therapeutically to treat, prevent, and/or reduce the risk of developing various diseases from various pathogens, such as a cancerous tumor. Methods disclosed herein can include methods of treating cancer in a subject including administering peptide epitopes, variants thereof, mimotopes, and mimotope variants thereof, which associate with an individual's MHC class I molecules and which are identified from VEL libraries based on the peptide's in vitro interaction, or lack thereof, with the unique subset of an individual's T cell repertoire, based on a lymphoproliferation assay of the individual's PBMCs.

In one embodiment, T cell proliferation assays involve the analysis of PBMCs from healthy individuals and patients (for example cancer patients) in both total cell proliferation assays by fluorescence-activated cell sorting (FACS) and cell phenotyping assays (for example, as described in NoeDominguez-Romero et al., (2014) Human Vaccines & Immunotherapeutics, 10(11):3201-3213, incorporated herein by reference, with mice spleen cells). In one embodiment, cell phenotyping involves determination of the subpopulations of proliferating T cells (e.g., CD4+ and CD8+ cells) using flow cytometry and intracellular cytokine staining (ICS) for IFIN-y assays. For example, PBMCs are analyzed by FACS either after 6 hours of stimulation or upon 3 days of incubation with phage-displayed variant epitopes showing superior antigenic properties in a cell proliferation assay described above compared with corresponding wild-type epitope and a non-related epitope. Also, a standard ELISPOT assay could be used as described (Gallou C. et al, Oncotarget. 2016 Aug. 5. doi: 10.18632/oncotarget.11086. [Epub ahead of print] hereby incorporated by reference herein in its entirety) or as described in Current Protocols in Immunology (Greene Pub. Associates, U.S., hereby incorporated by reference herein in its entirety) or any other Immunological Protocols known to one of skill. In one embodiment, randomly selected phage-displayed variant epitopes/mimotopes can be used as antigens (10⁷-10′ particles/well) or synthetic peptides (10⁻⁶M) randomly (in silico) selected from epitope or mimotope libraries described herein. In one example, 1000 randomly selected phage phage-displayed variant epitopes/mimotopes from an epitope derived VEL library bearing a complexity of 8000 individual members are screened in assays, including a cell proliferation assay of PBMCs from a patient. However, the number of phage/peptides randomly selected phage can vary from 1 or up to 5, or up to 10, 20, 50, 100, 200, 250, 400, 500, 750, 1000, 2000, 4,000, or higher. Similarly, screening of libraries (phage or peptide or otherwise) in the methods disclosed herein can comprise random selection of individual library members or non random selection of individual library members, and can include as few as one member, to as many as up to and including 10%, 20%, 30%, 40%, 50% 60%, 70%, 80%, 90% to 100% of the individual library members.

Methods disclosed herein further comprise treating a disease or disorder of an individual by administering a composition having one or more of these isolated peptides epitopes, variants thereof, mimotopes, and mimotope variants thereof, where the epitope is from an antigen related to the disease or condition. In one embodiment, the antigen is the tumor associated antigen survivin, an oncogenic inhibitor of apoptosis. In one aspect, the epitope of the survivin antigen is an amino acid sequence corresponding to a survivin CTL epitope, such as the survivin-derived H-2D^{d}-restricted wild-type CTL epitope, GWEPDDNPI (SEQ ID NO: 2). In some embodiments, VELs containing CTL-derived epitopes of survivin can be based, for example, on the epitope GWXPXDXPI (SEQ ID NO:1), where X is any one of the 20 naturally occurring amino acids or derivatives thereof.

In another embodiment, the mimotope is the wild type peptide sequence AGPAAAAAL (SEQ ID NO: 35). Encompassed in the methods herein are VEL libraries based on said the wild type peptide sequence AGPAAAAAL (SEQ ID NO: 35). Preferably encompassed are two types of mimotope VEL libraries which have been generated based on the amino acid sequences of this mimotope: one library having 3 mutated positions (AGPXAXAXL (SEQ ID NO: 3)) where X is any amino acid, and the second library having 5 mutated positions (PGSD) (5X library) (A[G/F]PXXXXX[L/M], (SEQ ID NO: 34)) where X is any amino acid.

Genetic variability of many tumor-related antigens and pathogen variable antigens can result in the selection of mutated epitope variants in the patient which are able to escape control by immune responses. This can be a major obstacle to treatment strategies against cancers and infection by certain pathogens. Preferable embodiments herein relate to the characterization of peptides from variable epitope libraries, which are derived from tumor antigens, pathogen antigens, and other disease-related antigens, preferably peptides able to bind MHC Class I molecules, with respect to their ability to interact with PBMC, especially CTLs, from an individual, in order to select peptides to administer to the individual which are effective to treat the disease or disorder afflicting the individual. Treatment of a disease or disorder afflicting the individual encompasses any amelioration of the disease or disorder, or symptoms thereof, whether temporary or permanent.

In a further embodiment, a subsequent VEL library is generated as described herein, based on the amino acid sequence of one or more of the peptide(s) of the vaccine or therapeutic agent previously administered to a patient. in one embodiment a cancer patient. In one aspect, a subsequent VEL library contains a library of peptides where anywhere any one or more amino acid positions of a peptide previously administered as a therapeutic agent and/or as a vaccine is varied by substitution in the amino acid sequence of the previously administered peptide of any amino acid at one or up to two, or three or four or five or six or seven amino acid positions of the peptide. Preferably the amino acid at each of the two anchor positions is not altered in one embodiment. Random clones from the subsequent VEL library are tested for their ability to stimulate proliferation of the patient's PBMCs as described herein. This combination of method steps involving the use of a subsequent VEL as described herein, allows for monitoring of the T-cell immune responses of patient who has been and/or is continuing to be treated with administration of a specified peptide.

In another similar aspect of monitoring, instead of using a subsequent VEL generated based on variants of a previously administered peptide as described above, the subsequent VEL is generated based on a mimotope as described above. The VELs generated based on mimotopes can be used to monitor immune responses induced in individuals vaccinated by any type of vaccine, because this type of VELs bearing mimotope libraries are generated independently, without any previous information on the nature of vaccine immunogen. That is, a subsequent VEL library contains a library of peptides where anywhere any one or more amino acid positions of a mimotope of a peptide previously administered as a therapeutic agent and/or as a vaccine is varied by substitution in the amino acid sequence of a mimotope of the previously administered peptide of any amino acid at one or up to two, or three or four or five or six or seven amino acid positions of the mimotope peptide. Preferably the amino acid at each of the two anchor positions is not altered in one embodiment. Random clones from the subsequent VEL library are tested for their ability to stimulate proliferation of the patient's PBMCs as described herein. This combination of method steps involving the use of a subsequent VEL as described herein, allows for monitoring of the T-cell immune responses of patient who has been and/or is continuing to be treated with administration of a specified peptide.

A peptide composition that includes peptide epitopes associated with a disease or disorder is referred to as a variable epitope library (VEL). VELs also can include variants of the epitope, mimotopes of the epitope, and variants of the mimotope. Preferably, the peptide epitopes are epitopes that associate or bind to MHC class I molecules, and range from about 7 to about 12 amino acids (AA) or amino acid residues in length, and are typically 9 amino acids long. For example, the peptides of a VEL can be P₁P₂P₃ . . . Pᵣᵢ, where the numbers represent positions (P) of the various wild type amino acids, and where “n” represents the total polypeptide length and the position of the last amino acid. In various embodiments disclosed herein, at least one amino acid and as many as 72% ( 5/7) of wild type amino acid residues and as few as 16% ( 2/12) can be randomly replaced by any of the 20 naturally occurring amino acid residues. As one of skill in the art would readily, VELs and VEL compositions are neither natural products nor naturally occurring, and VELs and VEL compositions are made-up of polypeptides that are neither natural products nor naturally occurring. VELs can contain nucleic acid sequence molecules comprising from about 20 to about 200 individual nucleotides that encode the variable epitope polypeptides. In other embodiments, VELs can contain one or more polypeptide molecules where from about 10% to about 50% of the total amino acids of the one or more polypeptide molecules are variable amino acids (replaced by any of the 20 naturally occurring amino acid residues or a derivative of a naturally occurring amino acid). In other embodiments, VELs can contain one or more polypeptides in which from about 20% to about 50% of the total amino acids of the one or more peptides are variable amino acids. In certain embodiments, VELs can contain one or more polypeptides in which from about 30% to about 50% of the total amino acids of the one or more peptides are variable amino acids. In yet other embodiments, VELs can contain one or more polypeptides in which from about 20% to about 40% of the total amino acids of the one or more peptides are variable amino acids.

For example, VELs and VEL vaccine compositions disclosed herein can be composed of a 9mer, Pi P₂ P₃ P4 P5 P6 P7 P8P9, that can be represented as PiP₂ P₃X₄ X₅X₆ P7P8 Pg, where X can be any of the 20 naturally occurring amino acids or derivatives of a naturally occurring amino acid, and where P can be an amino acid that is the same amino acid as that of the wild type epitope at that position, preferably anchor residues.

The complexities of VELs can range from a VEL composed of 20 epitope variants or mimotope variants, where only one wild-type amino acid residue is replaced in the epitope or mimotope by a random amino acid (e.g., 20 total peptides in the VEL), and up to about 20⁷ epitope variants, where several amino acid residues are mutated. In some embodiments, the complexities of VELs can range from about 20 different amino acids to about 20², or 20³ or 20⁴ different amino acids, depending on the number of variable amino acids, as one of skill in the art would recognize and understand based on the present disclosure and common knowledge. A VEL-based peptide can represent antigenic diversity observed during the course of cancer or other disease, including resulting from an infection with a pathogen. Use of VEL immunogens as disclosed herein permits the generation of novel prophylactic and therapeutic vaccines and treatments capable of inducing a broad range of protective immune responses before the appearance of mutated epitopes (very early stages of cancer or before pathogen infection) or when the amounts of mutated epitopes are low (early stages of cancer or pathogen infection and/or disease progression). Alternatively VELs and VEL compositions can be used prophylactically and/or therapeutically to treat, mid or late stage cancers and established diseases from various pathogens. The methods encompassing VEL-based peptides and libraries are particularly useful in the treatment of patients having later or advanced stages of cancer and/or having solid tumors, as such patients often display an immunotolerance to the cancer/tumor. The immunotolerance can be to the original or primary tumor, or to mutated forms of the original or primary tumor. Staging systems include the TNM staging system, as well as staging systems that are specific to a particular type of cancer. The TMN staging system is the most widely used system where the “T” refers to the size and extent of the main tumor (i.e. the primary tumor), the “N” refers to the number of nearby lymph nodes that have cancer, and the “M” refers to whether the cancer has metastasized, i.e., that the cancer has spread from the primary tumor to other parts of the body.

VELs are preferably generated based on a defined antigen of the cancer or pathogen or disease-related antigen-derived cytotoxic T lymphocyte (CTL). The epitopes are preferably derived from antigenically variable or relatively conserved regions of the protein antigen. Alternatively, VELs can be generated based on up to 50 amino acid long peptide regions of antigens containing clusters of epitopes. An individual VEL can contain: [1] a CTL epitope and variants of one CTL epitope; [2] variants of several different CTL epitopes; [3] mimotopes of said one CTL epitope or of said several different CTL epitopes; [4] variants of said mimotope or mimotopes [5] any combination of [1] to [4]. In one embodiment a VEL is generated based on a CTL peptide epitope of 7-12 amino acids selected from a tumor antigen or from an antigenically variable or a relatively conserved region of a pathogen- or disease-related protein without a prior knowledge of the existence of epitopes in these peptide regions. Candidate CTL epitopes can be selected from scientific literature or from public databases. A VEL comprising a CTL epitopes, mimotope thereof, epitope variants thereof and/or mimotope variants thereof, in VELs are important since the escape from protective CTL responses is an important mechanism for immune evasion by cancer cells and by many pathogens, for example HIV and SIV.

VELs can take the form of DNA constructs, recombinant polypeptides or synthetic peptides and can be generated using standard molecular biology or peptide synthesis techniques, as discussed below. For example to generate a DNA fragment encoding peptide variants of a particular epitope, a synthetic 40-70 nucleotide (nt) long oligonucleotide (oligo) carrying one or more random amino acid-coding degenerate nucleotide triplet(s) may be designed and produced. The epitope-coding region of this oligo (oligol) may contain non-randomized 9-15 nucleotide segments at 5′ and 3′ flanking regions that may or may not encode natural epitope-flanking 3-5 amino acid residues. Then, 2 oligos that overlap at 5′ and 3′ flanking regions of oligol and carry nucleotide sequences recognized by hypothetical restriction enzymes A and B, respectively, may be synthesized and after annealing reaction with oligol used in a PCR. This PCR amplification will result in mutated epitope library-encoding DNA fragments that after digestion with A and B restriction enzymes may be combined in a ligation reaction with corresponding bacterial, viral or eukaryotic cloning/expression vector DNA digested with the same enzymes. Ligation mixtures can be used to transform bacterial cells to generate the VEL and then expressed as a plasmid DNA construct, in a mammalian virus or as a recombinant polypeptide. This DNA can also be cloned in bacteriophage, bacterial or yeast display vectors, allowing the generation of recombinant microorganisms.

In a similar manner, DNA fragments bearing 20-200 individual nucleotides can encode various combinations of different mutated epitope variants or mimotope variants. These nucleic acid molecules can be created using sets of long overlapping oligos and a pair of oligos carrying restriction enzyme recognition sites and overlapping with adjacent epitope-coding oligos at 5′ and 3′flanking regions. These oligos can be combined, annealed and used in a PCR assembly and amplification reactions. The resulting DNAs may be similarly cloned in vectors, e.g., mammalian virus vectors, and expressed as recombinant peptides or by recombinant microorganisms. The peptides may be used individually in immunotherapy or may be combined and used as a mixture of peptides.

In one example, synthetic peptide VELs varying in length from 7 to 12 amino acid residues may be generated by solid phase Fmoc peptide synthesis technique where in a coupling step equimolar mixtures of all proteogeneic amino acid residues may be used to obtain randomized amino acid positions. This technique permits the introduction of one or more randomized sequence positions in selected epitope sequences and the generation of VELs with complexities of up to 10⁹, though preferably ranging from about 100 to 1000.

Peptide variants of an epitope or a mimotope based on VELs can be assessed and selected based on their interaction with an individual's PBMC, which are a source of CTLs. Thus selected peptide variants of an epitope or a mimotope can be useful for inducing immune responses, especially CTL response against tumors and pathogens with antigenic variability as well as may be effective in modulating allergy, inflammatory and autoimmune diseases. In one embodiment, pharmaceutical compositions containing one or more VEL derived, selected peptide variants of a CTL epitope or a mimotope may be formulated with a pharmaceutically acceptable carrier, excipient and/or adjuvant, and administered to the individual, such as a non-human animal or a human patient. These pharmaceutical compositions can be administered to a subject, such as a human, therapeutically or prophylactically at dosages ranging from about 100 lig to about 1 mg of isolated peptides. Compositions containing VELs including nucleic acid sequences of the above peptides can be administered to a subject, such as a human, therapeutically or prophylactically at dosages ranging from about 1×10¹⁰ to about 5×10⁵ CFU of bacteriophage particles. In some embodiments, these pharmaceutical peptide or nucleic acid compositions administered to a human subject can reduce onset of a disease such as a cancer (e.g., a malignant cancer such as a malignant tumor involving survivin) and/or can treat a disease already existing in the human subject (e.g., a cancerous malignancy involving survivin). Other approaches for the construction of VELs, expression and/or display vectors, optimum pharmaceutical composition, routes for peptide or nucleic acid delivery and dosing regimens capable of inducing prophylactic and/or therapeutic benefits may be determined by one skilled in the art based on the present disclosure. For example, compositions containing these pharmaceutical peptide or nucleic acid compositions can be administered to a subject as a single dose application, as well as a multiple dose (e.g., booster) application. Multiple dose applications can include, for example, administering from about 1 to about 25 total dose applications, with each dose application administered at one or more dosing intervals that can range from about 7

days to about 14 days (e.g., weekly). In some embodiments, dosing intervals can be administered daily, two times daily, twice weekly, weekly, monthly, bi-monthly, annually, or bi-annually, depending on the particular needs of the subject and the characteristics of the condition being treated or prevented (or reducing the risk of getting the condition), as would be appreciated by one of skill in the art based on the present disclosure.

The skilled artisan will realize that in alternative embodiments, less than the 20 naturally occurring amino acids may be used in a randomization process. For example, certain residues that are known to be disruptive to protein or peptide secondary structure, such as proline residues, may be less preferred for the randomization process. VELs may be generated with the 20 naturally occurring amino acid residues or with some subset or derivatives of the 20 naturally occurring amino acid residues. In various embodiments, in addition to or in place of the 20 naturally occurring amino acid residues, the VELs may contain at least one modified amino acid.

### Combinatorial Libraries

Combinatorial libraries of such compounds or of such targets can be categorized into three main categories. The first category relates to the matrix or platform on which the library is displayed and/or constructed. For example, combinatorial libraries can be provided (i) on a surface of a chemical solid support, such as micro-particles, beads or a flat platform; (ii) displayed by a biological source (e.g., bacteria or phage); and (iii) contained within a solution. In addition, three dimensional structures of various computer generated combinatorial molecules can be screened via computational methods.

The third category of combinatorial libraries relates to the method by which the compounds or targets are synthesized, such synthesis is typically effected by: (i) in situ chemical synthesis; (ii) in vivo synthesis via molecular cloning; (iii) in vitro biosynthesis by purified enzymes or extracts from microorganisms; and (iv) in silico by dedicated computer algorithms.

Combinatorial libraries indicated by any of the above synthesis methods can be further characterized by: (i) split or parallel modes of synthesis; (ii) molecules size and complexity; (iii) technology of screening; and (iv) rank of automation in preparation/screening.

### Expression of Peptides

In certain embodiments, it may be preferred to make and use an expression vector that encodes and expresses a particular VEL. Gene sequences encoding various polypeptides or peptides may be obtained from GenBank and other standard sources, as disclosed above. Expression vectors containing genes encoding a variety of known proteins may be obtained from standard sources, such as the American Type Culture Collection (Manassas, Va.). For relatively short VELs, it is within the skill in the art to design synthetic DNA sequences encoding a specified amino acid sequence, using a standard codon table, as discussed above. Genes may be optimized for expression in a particular species of host cell by utilizing well-known codon frequency tables for the desired species.

Regardless of the source, a coding DNA sequence of interest can be inserted into an appropriate expression system. The DNA can be expressed in any number of different recombinant DNA expression systems to generate large amounts of the polypeptide product, which can then be purified and used in various embodiments of the present disclosure.

Examples of expression systems known to the skilled practitioner in the art include bacteria such as E. coli, yeast such as Pichia pastoris, baculovirus, and mammalian expression systems such as in Cos or CHO cells. Expression is not limited to single cells, but may also include protein production in genetically engineered transgenic animals, such as mice, rats, cows or goats.

The nucleic acid encoding a peptide may be inserted into an expression vector by standard subcloning techniques. An E. coli expression vector may be used which produces the recombinant polypeptide as a fusion protein, allowing rapid affinity purification of the peptide. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6XHis system (Qiagen, Chatsworth, Calif.).

Some of these systems produce recombinant polypeptides bearing only a small number of additional amino acids, which are unlikely to affect the activity or binding properties of the recombinant polypeptide. For example, both the FLAG system and the 6XHis system add only short sequences, both of which have no adverse effect on folding of the polypeptide to its native conformation. Other fusion systems are designed to produce fusions wherein the fusion partner is easily excised from the desired peptide. In one embodiment, the fusion partner is linked to the recombinant peptide by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

The expression system used may also be one driven by the baculovirus polyhedron promoter. The gene encoding the polypeptide may be manipulated by standard techniques in order to facilitate cloning into the baculovirus vector. One baculovirus vector is the pBlueBac vector (Invitrogen, Sorrento, Calif.). The vector carrying the gene for the polypeptide is transfected into Spodoptera frugiperda (Sf9) cells by standard protocols, and the cells are cultured and processed to produce the recombinant protein.

In one embodiment expression of a recombinant encoded peptide comprises preparation of an expression vector that comprises one of the isolated nucleic acids under the control of, or operatively linked to, one or more promoters. To bring a coding sequence “under the control of” a promoter, the 5′ end of the transcription initiation site of the transcriptional reading frame is positioned generally from about 1 to about 50 nucleotides “downstream” (3′) of the chosen promoter. The “upstream” promoter stimulates transcription of the DNA and promotes expression of the encoded recombinant protein.

Many standard techniques are available to construct expression vectors containing the appropriate nucleic acids and transcriptional/translational control sequences in order to achieve peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors. Non-limiting examples of prokaryotic hosts include E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which may be used by the microbial organism for expression of its own proteins.

In addition, phage vectors containing replicon and control sequences that are compatible with the host microorganism may be used as transforming vectors in connection with these hosts. For example, the phage lambda GEMTM-11 may be utilized in making a recombinant phage vector which may be used to transform host cells, such as E. coli LE392.

Further useful vectors include pIN vectors and pGEX vectors, for use in generating glutathione S transferase (GST) soluble fusion proteins for later purification and separation or cleavage. Other suitable fusion proteins are those with I3-galactosidase, ubiquitin, or the like. Preferable promoters for use in recombinant DNA construction include the 13-lactamase (penicillinase), lactose and tryptophan (trp) promoter systems. However, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid already contains the trpl gene which provides a selection marker for a mutant strain of yeast lacking the ability to grow in tryptophan, for example ATCC No. 44076 or PEP4-1. The presence of the trp/lesion as a characteristic of the yeast host cell genome then provides an effective environment for detecting transformation by growth in the absence of tryptophan.

Suitable promotor sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a preferable insect system, Autographa californica nuclear polyhidrosis virus (AcNPV) is used as a vector to express foreign genes. The virus grows in Spodoptera frugiperda cells. The isolated nucleic acid coding peptide sequences are cloned into non-essential regions (e.g., polyhedrin gene) of the virus and placed under control of an AcNPV promoter (e.g., polyhedrin promoter). Successful insertion of the coding sequences results in the inactivation of the polyhedrin gene and production of non-occluded recombinant virus (e.g., virus lacking the proteinaceous coat coded for by the polyhedrin gene). These recombinant viruses are then used to infect Spodoptera frugiperda cells in which the inserted nucleic acid coding the peptide sequences is expressed.

Examples of preferable mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted peptide encoding sequences, or modifies and processes the peptide product in the specific fashion desired.

Different host cells have characteristic and specific mechanisms for the post-translational processing and modification of proteins. Appropriate cells lines or host systems may be chosen to ensure the correct modification and processing of the foreign peptide expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter) as known in the art.

A number of viral based expression systems may be utilized, for example, commonly used promoters are derived from polyoma, Adenovirus 2, and most frequently Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the Hind III site toward the Bgl I site located in the viral origin of replication.

In one example where an adenovirus is used as an expression vector, the peptide coding sequences may be ligated to an adenovirus transcription/translation control complex (e.g., the late promoter and tripartite leader sequence). This chimeric gene may then be inserted in the adenovirus genome by in vitro or in vivo recombination. Insertion in a non-essential region of the viral genome (e.g., region El or E3) will result in a recombinant virus that is viable and capable of expressing the peptides in infected hosts.

Specific initiation signals known in the art may also be required for efficient translation of the claimed isolated nucleic acid encoding the peptide sequences. One of ordinary skill in the art would readily be capable of determining this and providing the necessary signals

A number of selection systems may be used, including but not limited to, the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, antimetabolite resistance may be used as the basis of selection for dihydrofolate reductase (DHFR), which confers resistance to methotrexate; xanthineguanine phosphoribosyl transferase (gpt), which confers resistance to mycophenolic acid; neomycin (neo), that confers resistance to the aminoglycoside G-418; and hygro, which confers resistance to hygromycin. These and other selection genes may be obtained in vectors from, for example, ATCC or may be purchased from a number of commercial sources known in the art (e.g., Stratagene, La Jolla, Calif.; Promega, Madison, Wis.).

Where substitutions of a pathogen- or disease-related epitope or mimotope thereof are desired, the nucleic acid sequences encoding the substitutions may be manipulated by well-known techniques, such as site-directed mutagenesis or by chemical synthesis of short oligonucleotides followed by restriction endonuclease digestion and insertion into a vector, by PCR based incorporation methods, or any similar method known in the art.

### Protein Purification

In certain embodiments the peptide(s) may be isolated or purified. Protein purification techniques are well known to those of skill in the art. These techniques involve, at one level, the homogenization and crude fractionation of the cells to peptide and non-peptide fractions. The peptide(s) of interest may be further purified using chromatographic and electrophoretic techniques to achieve partial or complete purification (or purification to homogeneity). Analytical methods well suited to the preparation of a pure peptide are ion-exchange chromatography, gel exclusion chromatography, polyacrylamide gel electrophoresis, affinity chromatography, immunoaffinity chromatography and isoelectric focusing. An efficient method of purifying peptides is fast performance liquid chromatography (FPLC) or even HPLC.

A purified peptide is intended to refer to a composition, isolatable from other components, wherein the peptide is purified to any degree. An isolated or purified polypeptide or peptide, therefore, also refers to a polypeptide or peptide free from the environment from which it originated. Generally, “purified” will refer to a peptide composition that has been subjected to fractionation to remove various other components. Where the term “substantially purified” is used, this designation will refer to a composition in which the peptide forms the major component of the composition, such as constituting about 50%, about 60%, about 70%, about 80%, about 90%, about 95%, or more of the peptides in the composition. Various methods for quantifying the degree of purification of the peptide are known to those of skill in the art in light of the present disclosure.

Various techniques suitable for use in peptide purification are contemplated herein and are well known. There is no general requirement that the peptide always be provided in their most purified state. Indeed, it is contemplated that less substantially purified products will have utility in certain embodiments. In another embodiment, affinity chromatography may be required and any means known in the art is contemplated herein.

### Formulations and Routes for Administration to Subjects

Where clinical applications are contemplated, it will be necessary to prepare pharmaceutical compositions (e.g., VEL peptide compositions) in a form appropriate for the intended application. Generally, this will entail preparing compositions that are essentially free of impurities that could be harmful to human or animal subjects.

Preferably, the peptide compositions comprise salts and buffers to render the peptides stable and allow for interaction with target cells. Aqueous compositions may comprise an effective amount of peptide dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the polypeptides of the present disclosure, its use in therapeutic compositions is contemplated. Supplementary active ingredients also can be incorporated into the compositions.

The active peptide compositions instantly disclosed include classic pharmaceutical preparations. Administration of these compositions according to the present disclosure will be via any common route. This includes oral, nasal, buccal, rectal, vaginal, topical, orthotropic, intradermal, subcutaneous, intramuscular, intraperitoneal, intraarterial or intravenous injection. Such compositions normally would be administered as pharmaceutically acceptable compositions, as described above.

The active peptide compounds also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent needed for easy application via syringe. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In certain examples, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. Regarding sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCI solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The VELs and VEL peptide compositions of the present disclosure may also be used in conjunction with targeted therapies, including but not limited to, therapies designed to target tumors and the cells

underlying the tumor. Many different targeted therapies have been approved for use in cancer treatment. For example, these therapies can include hormone therapies, signal transduction inhibitors, gene expression modulator, apoptosis inducer, angiogenesis inhibitor, immunotherapies, and toxin delivery molecules. Additionally, cancer vaccines and gene therapy can be considered targeted therapies because they interfere with the growth of specific cancer cells (e.g., breast cancer cells).

### Cell Proliferation Assays

Lymphocyte proliferation assay' comprises isolating peripheral blood mononuclear cells (PBMCs), placing 100,000 of the cells in each well of a 96-well plate with or without various stimuli, and allowing the cells to proliferate for six days at 37° C. in a CO₂ incubator. The amount of proliferation is detected on the sixth day by adding radioactive ³H (tritiated) thymidine for six hours, which is incorporated into the newly synthesized DNA of the dividing cells. The amount of radioactivity incorporated into DNA in each well is measured in a scintillation counter and is proportional to the number of proliferating cells, which in turn is a function of the number of lymphocytes that were stimulated by a given antigen to enter the proliferative response. The readout is counts per minute (cpm) per well.

### Detailed Lymphocyte Proliferation Assay'

Briefly, 10 m1 of heparinized venous blood was drawn from each study subject. For WB assay, 1:5 and 1:10 dilutions were made with sterile RPMI 1640 medium (Sigma Chemical Company, MO, USA), supplemented with penicillin (100 !Wm!), streptomycin (0.1 mg/ml), L-glutamine (0.29 gm/I) and amphotericin B (5 mg/m1) and was seeded in 96-well flat bottom plates at 200 μl/well.

PBMC were isolated by Ficoll-Hypaque density centrifugation. A total of 2×10⁵ cells/well were cultivated in complete culture medium, supplemented with 10% Human AB serum. Cultures were stimulated either with candidate peptide (5 μg/ml), or PHA (5 μg/ml) as a positive control or PPD (5 μg/ml). Cells cultured under similar conditions without any stimulation served as the negative control. The cultures were set up in triplicates and incubated for 6 days at 37° C. in 5% CO₂ atmosphere. Sixteen hours before termination of cultures, 1 u.Ci of tritiated (³H) thymidine (Board of Radiation and Isotope Technology, MA, USA) was added to each well. The cells were then harvested onto glass fiber filters on

²https://www.hanc.infollabs/labresources/procedures/ACTGIMPAACT%20LAB%20Manual/Lymphocyte%20Profileration%20Assay.pdf
³Deenadayalan et al. Comparison of whole blood and PBMC assays for T-cell functional analysis BMC Research Notes 2013, 6:120
a cell harvester and allowed to dry overnight. 2 m1 of scintillation fluid (0.05 mg/ml POPOP and 4 mg/ml PPO in lit. of toluene) was added to each tube containing the dried filter discs and counted by using a liquid scintillation beta counter.

The proliferation was measured as uptake of tritiated thymidine by cells and expressed as stimulation index (SI) which was calculated as Stimulation Index=mean counts per minute with peptide/mean counts per minute without peptide.

### Interferon-y Measurement'

For quantification of IFN-y, in all 1:5 and 1:10 diluted blood and PBMC cell-free culture supernatants from lymphocyte proliferation assay were harvested after 6 days of in vitro stimulation with or without antigen stimuli and stored at −80° C. until assayed. IFN-y production was determined by standard ELISA technique using commercially available BD opt-EIA Kit (BD Biosciences, Franklin Lakes, N.J., USA) as per the manufacturer's instructions.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or an when used in conjunction with the term “comprising” in the claims and/or the specification may mean “one,” but it is also consistent with the meaning of “one or more,” “at least one,” and “one or more than one.” The use of the term or in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the feature in the context with which it is referred. The term “substantially” when referring to an amount, extent or feature (e.g., “substantially identical” or “substantially the same”)

⁴ Deenadayalan et al. BMC Res Notes. 2013; 6: 120.
includes a disclosure of “identical” or “the same” respectively, and this provides basis for insertion of these precise terms into claims below.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term or combinations thereof' as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non limiting exemplifications.

### WORKING EXAMPLES

### Working Example I

### Construction of Variable Epitope Libraries (VELs)

In order to avoid tumor escape, it is desirable to target a tumor antigen that is essential for tumor survival and expressed by tumors at high levels. One of these antigens is survivin, an oncogenic inhibitor-of-apoptosis protein, which is expressed at high levels in virtually all malignancies and is commonly referred to as a universal tumor antigen. Additionally, survivin-specific T-cell reactivity strongly correlates with tumor response and subject survival. In one embodiment of the present disclosure, phage display VELs and synthetic peptide VELs were generated based on the survivin-derived CTL epitope presented below:

GWXPXDXPI (SEQ ID NO: 1), where X is any of the 20 naturally occurring amino acids or derivatives thereof.

VELs were generated using the recombinant M13 phage display system based on the survivin-derived H-2Dd-restricted wild-type CTL epitope, GWEPDDNPI (SEQ ID NO:2), referred to as SWT. The recombinant phage display library comprising the wild-type survivin epitope is referred to as FSWT, and the recombinant phage display VEL comprising the variable epitopes of wild-type survivin is referred to as FSVL. Additionally, the synthetic peptide library comprising the wild-type survivin epitope is referred to as PSWT, and the synthetic peptide VEL comprising the variable epitopes of wild-type survivin is referred to as PSVL.

The epitope variants comprising the combinatorial VELs, were generated using degenerate oligonucleotides encoding a library of epitope variants with structural composition GWXPXDXPI, (SEQ ID NO:1), where X is any of 20 natural amino acids.

To generate the VELs, molecular biology procedures were carried out using standard protocols, including the use of restriction enzymes, Taq DNA polymerase, DNA isolation/purification kits, T4 DNA ligase and M13K07 helper phages. In order to express the survivin-derived wild-type CTL peptide epitope GWEPDDNPI (SEQ ID NO. 2) and epitope variant-bearing VELs on M13 phage surfaces as fusions with the major phage coat protein (cpVlll), the corresponding DNA fragments were generated by PCR and cloned in a pG8SAET phagemid vector. Briefly, two oligonucleotides (oligos): 5′-gtat attactgtgcgggttgggaaccagatgataatccaatatggggccagggaacc-3′ (SEQ ID NO:4) and degenerate 5′-gtatattactgtgcgggttgg NNKccaNNK gatNNKccaatatggggccagggaacc-3′ (SEQ ID NO:5), (N is g, a, t or c and, K is g or c nucleotide) were used in two separate PCRs with pair of primers carrying Nco I and Barn HI restriction sites; 5DAMP: 5′-tgatattcgtactcgagccatggtgtatattactgtgcg-3′ (SEQ ID NO:6) and 3DAMP: 5-atgattgacaaagcttggatccctaggttccctggcccca-3 (SEQ ID NO:7) were used to generate corresponding DNA fragments for their cloning in phagemid vectors using electroporation. Correct sequences were verified using standard automated sequencers.

The resulting recombinant phage clone expressing the wild type epitope and the VEL phage library carrying epitope variants, were rescued/amplified using M13KO7 helper phages by infection of E. coli TG1 cells and purified by double precipitation with polyethylene glycol (20% PEG/2.5 M NaCl). 87 phage clones were randomly selected from the VEL library, each expressing different epitope variants, and rescued/amplified from 0.8 mL of bacterial cultures using 96 well 1 mL round bottom blocks. The typical phage yields were 10¹⁰ to 10¹¹ colony forming units (CFU) per milliliter of culture medium. The DNA inserts of 27 phage clones from the VEL library were sequenced and the amino acid sequences of the peptides were deduced, as presented in Table 1 below.

TABLE 1
Sequences of survivin-derived SWT epitope variants

| Wild-type epitope SWT | G | W | E | P | D | D | N | P | I |
|---|---|---|---|---|---|---|---|---|---|
| Epitope Library | G | W | X^{x} | P | X | D | X | P | I |
| Epitope Variants | | | | | | | | | |
| 1 | — | — | F | — | L | — | A | — | — |
| 2 | — | — | L | — | N | — | Y | — | — |
| 3 | — | — | R | — | T | — | V | — | — |
| 4 | — | — | P | — | L | — | N | — | — |
| 5 | — | — | I | — | S | — | F | — | — |
| 6 | — | — | Q | — | T | — | E | — | — |
| 7b | — | — | T | — | K | — | D | — | — |
| 8 | — | — | D | — | L | — | I | — | — |
| 9 | — | — | Q | — | M | — | S | — | — |
| 10 | — | — | I | — | T | — | A | — | — |
| 12 | — | — | C | — | Y | — | T | — | — |
| 22 | — | — | N | — | S | — | L | — | — |
| 25 | — | — | V | — | T | — | L | — | — |
| 38 | — | — | H | — | L | — | N | — | — |
| 41 | — | — | N | — | F | — | G | — | — |
| 45 | — | — | O | — | L | — | O | — | — |
| 50 | — | — | A | — | N | — | N | — | — |
| 53 | — | — | V | — | D | — | Y | — | — |
| 58 | — | — | Q | — | V | — | R | — | — |
| 59 | — | — | E | — | T | — | H | — | — |
| 65 | — | — | C | — | Q | — | L | — | — |
| 73 | — | — | W | — | Q | — | E | — | — |
| 79 | — | — | F | — | L | — | V | — | — |
| 80 | — | — | V | — | Y | — | Y | — | — |
| 82 | — | — | R | — | V | — | P | — | — |
| 88 | — | — | T | — | I | — | R | — | — |
| Amino acid frequencies | | | 14/26 | | 12/26 | | 16/26 | | |

^{x}X-any of 20 natural amino acids.
bThe clones marked in grey were used as Ag in T-Cell assays.

The wild-type epitope SWT is SEQ ID NO:2; where the Epitope Library is SEQ ID NO:1; Where epitope variants 1, 2, 3, 4, 5, 6, 7b, 8, 9, 10, 12, 22, 25, 38, 41, 45, 50, 53, 58, 59, 65, 73, 79, 80, 82 and 88 are SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO: 30, SEQ ID NO:31, SEQ ID NO:32 and SEQ ID NO:33, respectively.

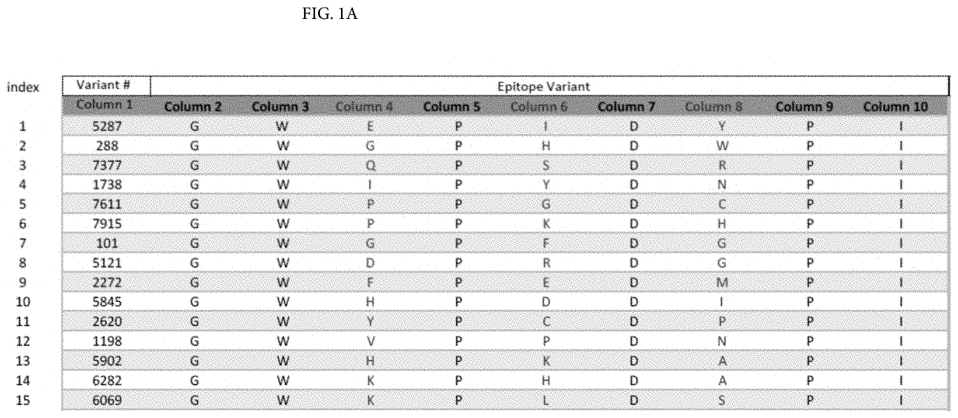

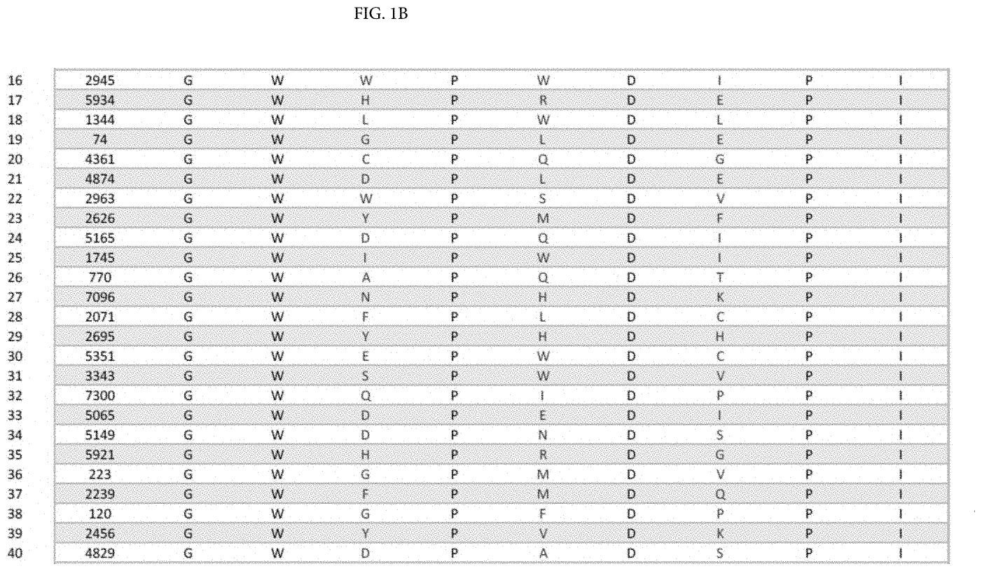

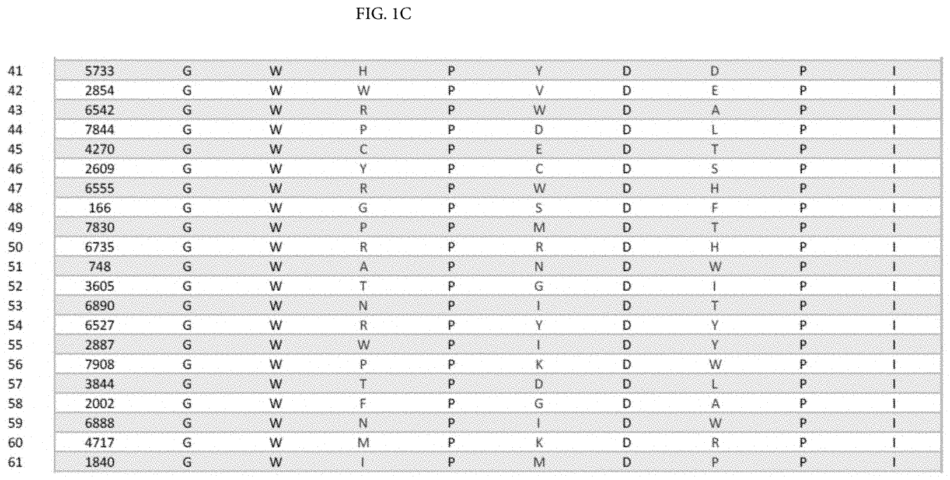

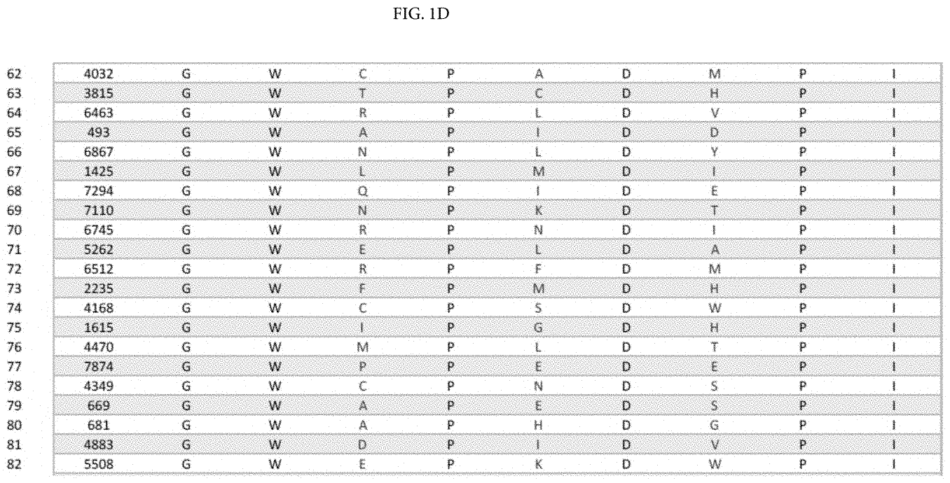

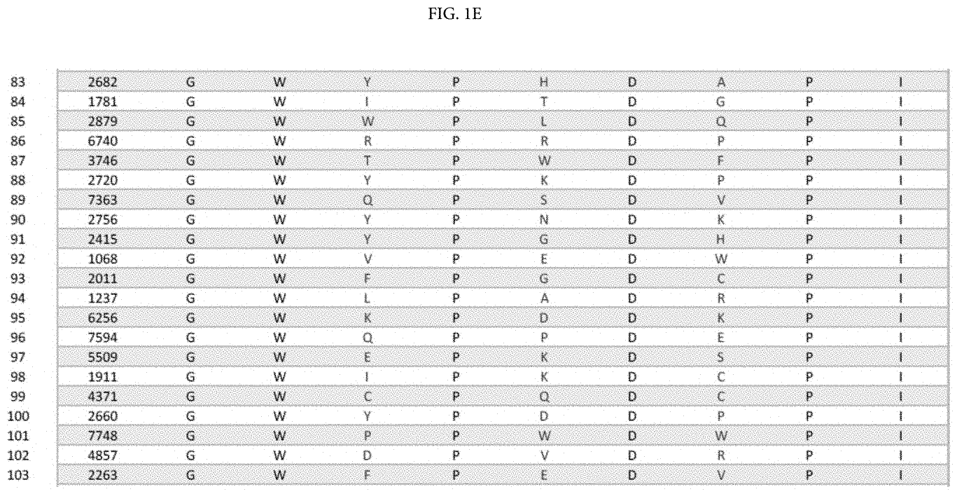

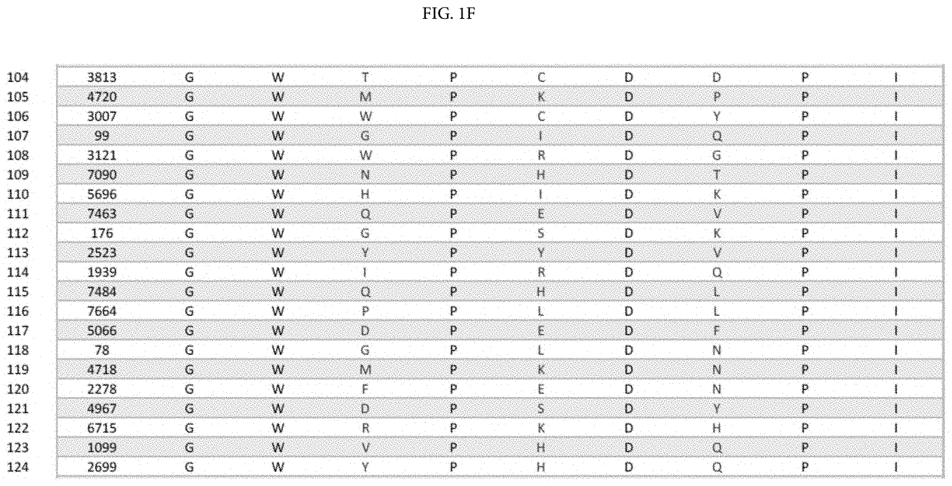

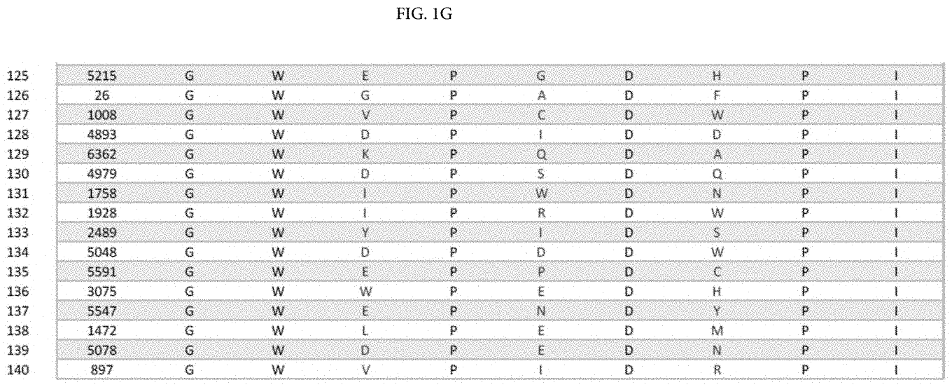

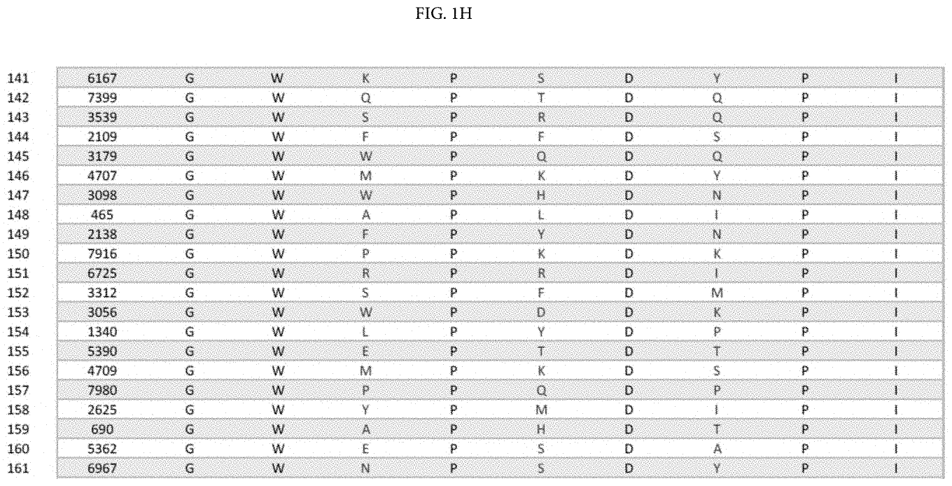

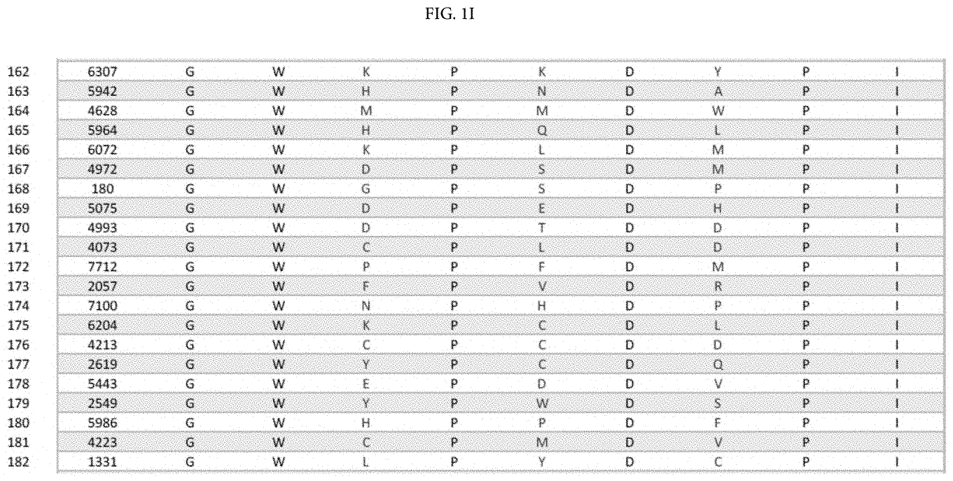

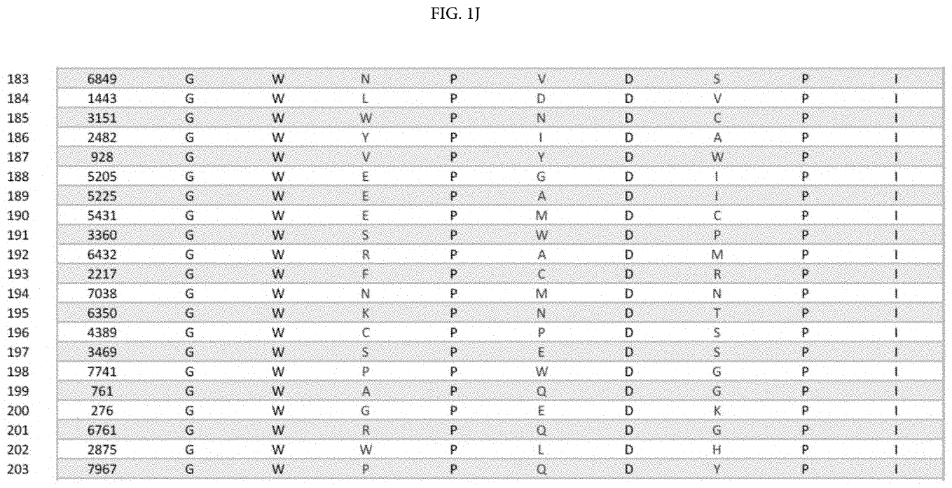

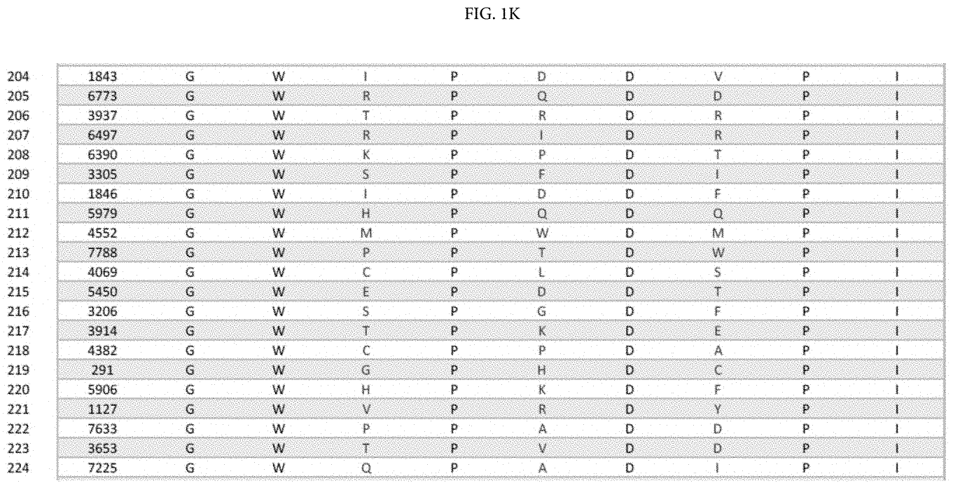

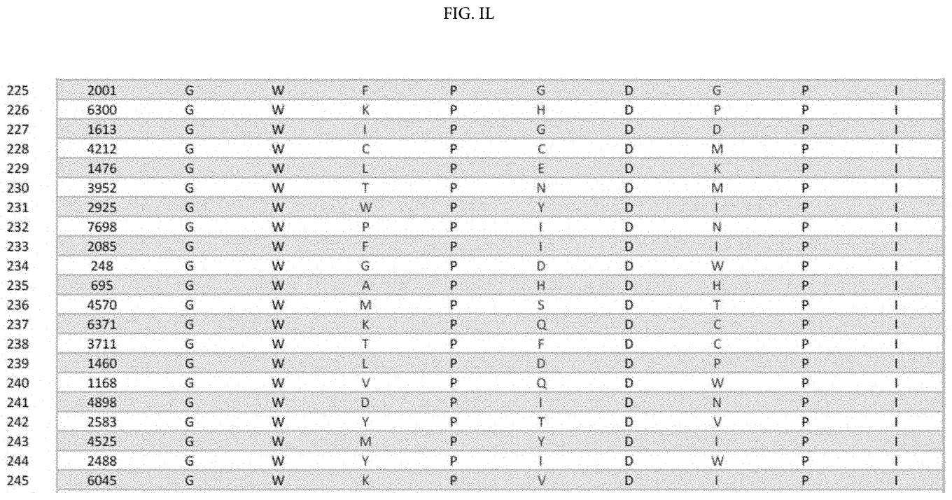

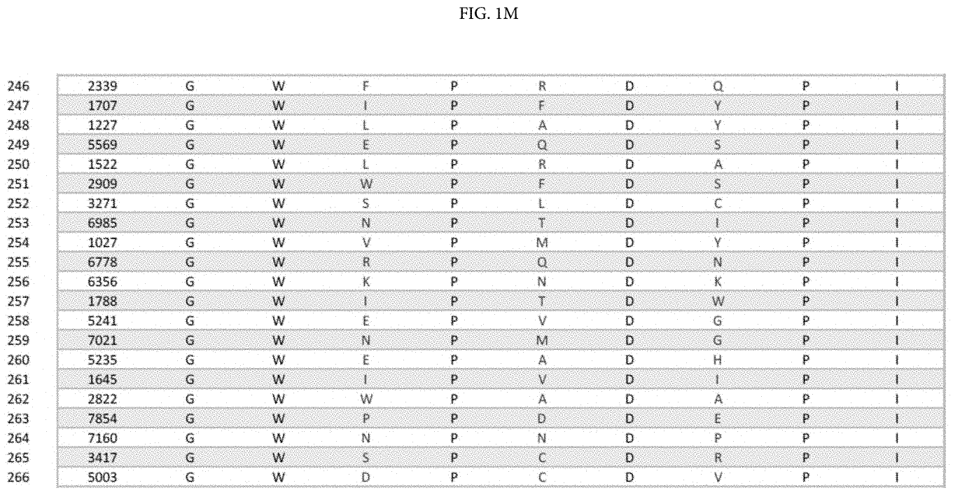

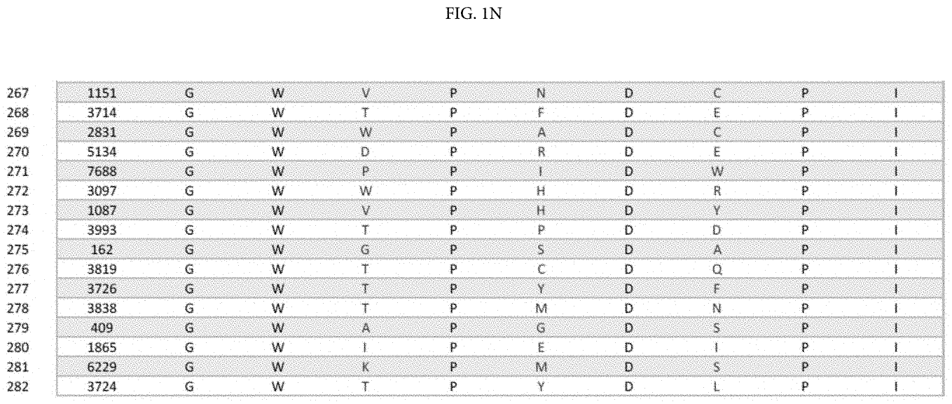

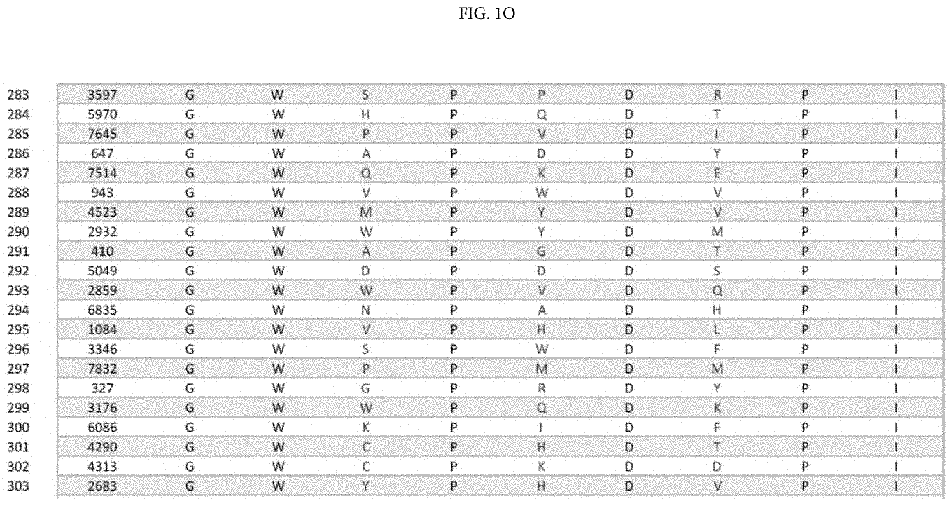

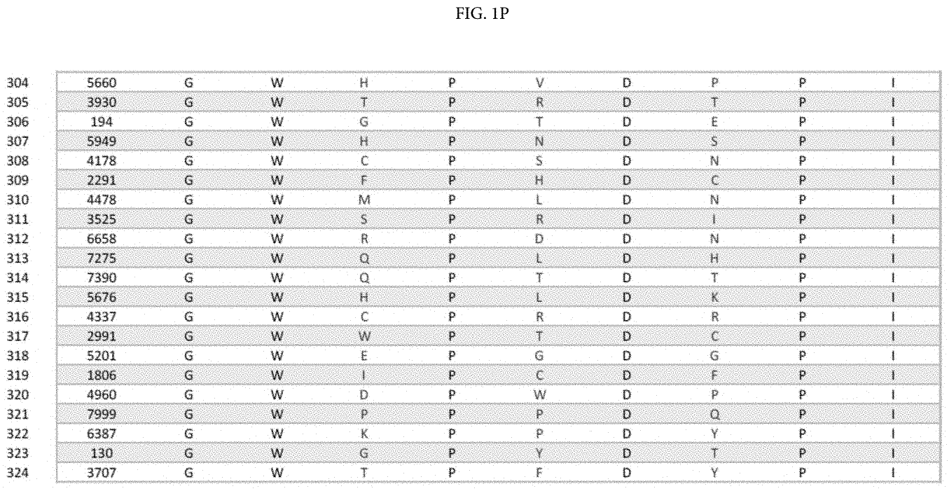

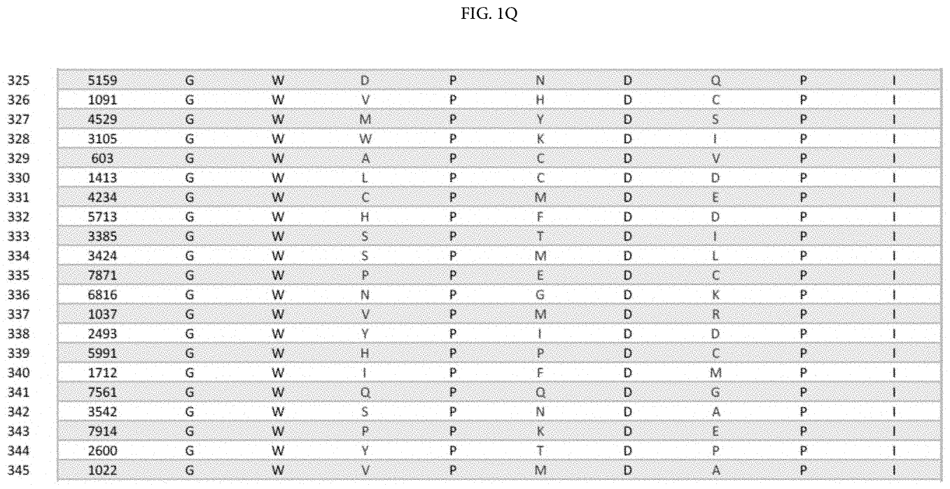

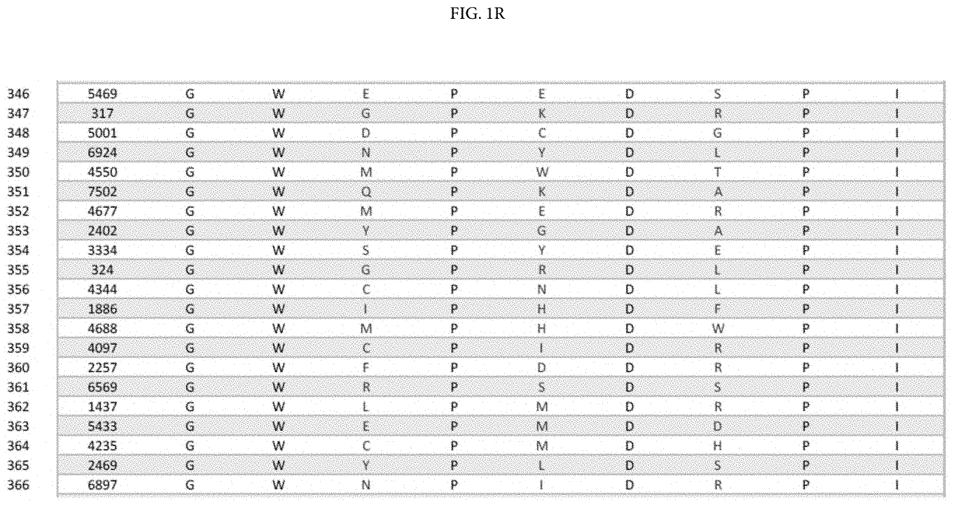

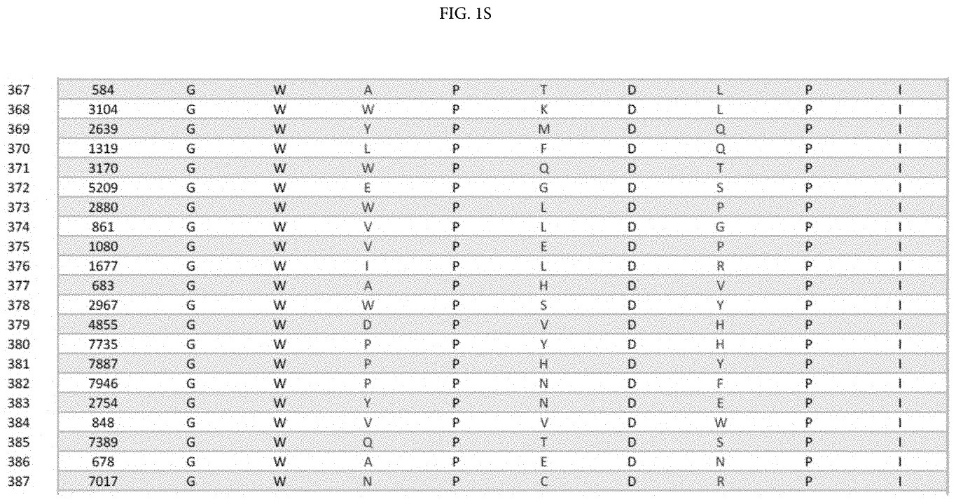

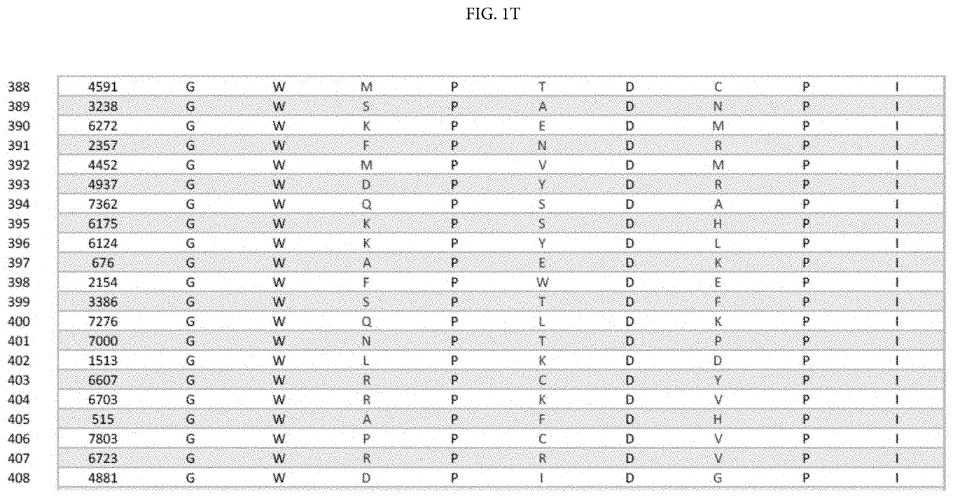

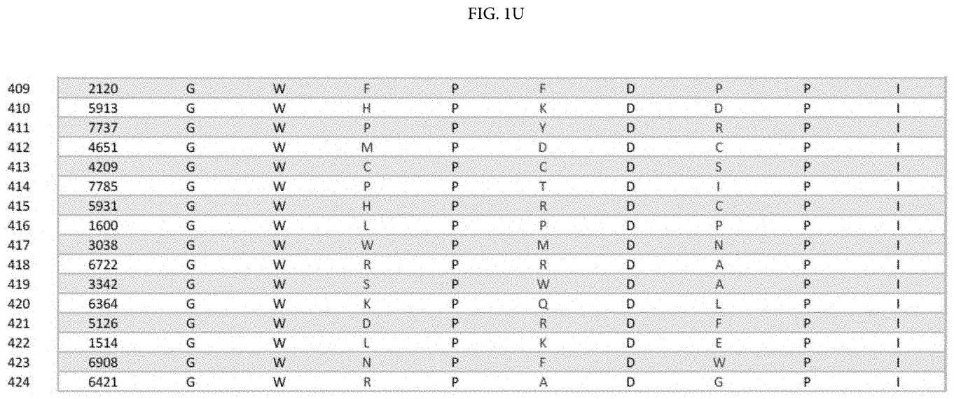

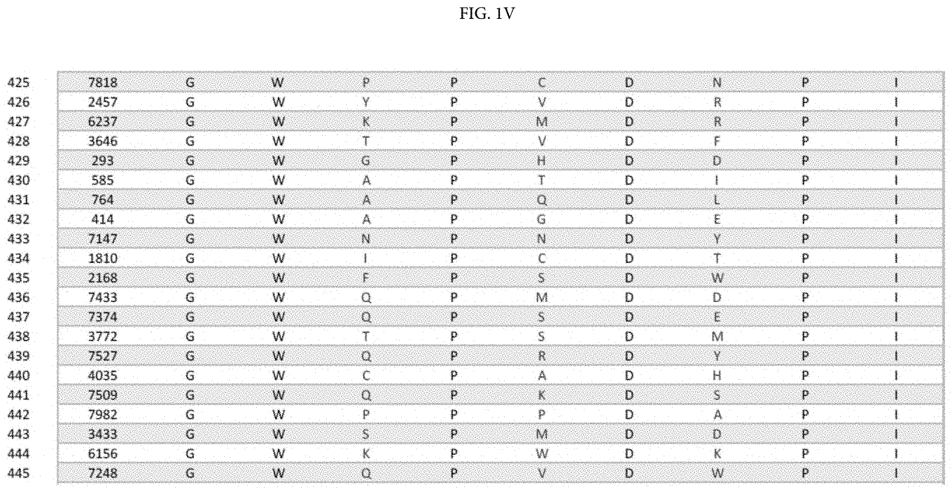

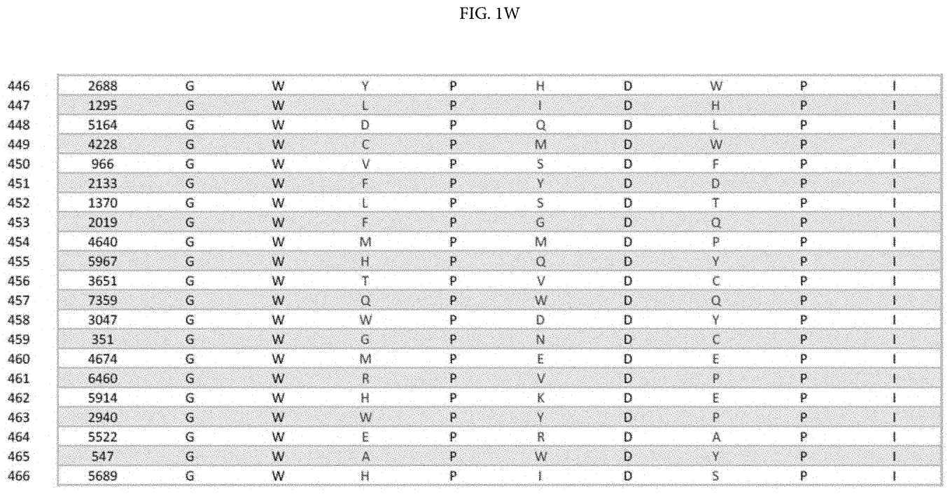

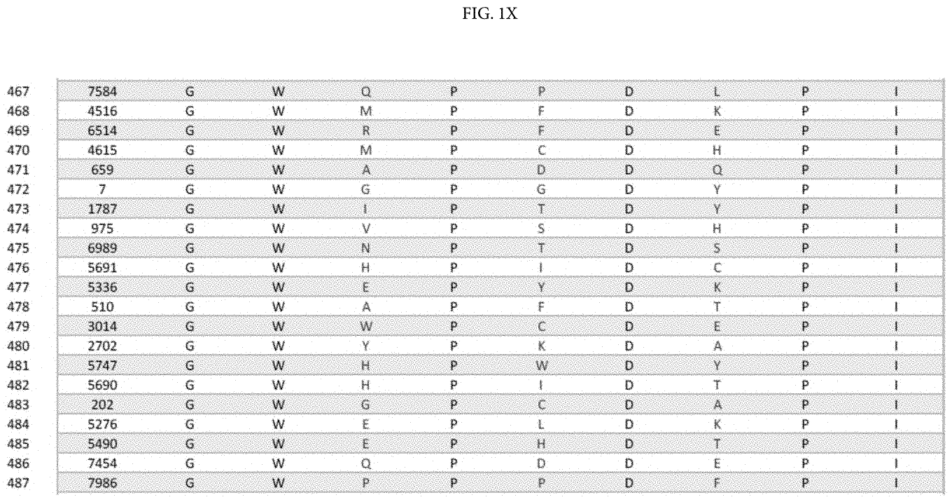

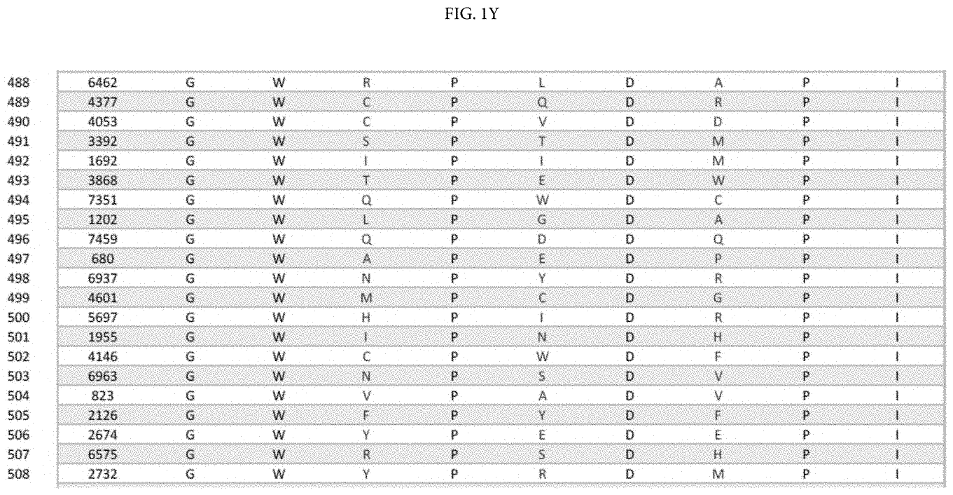

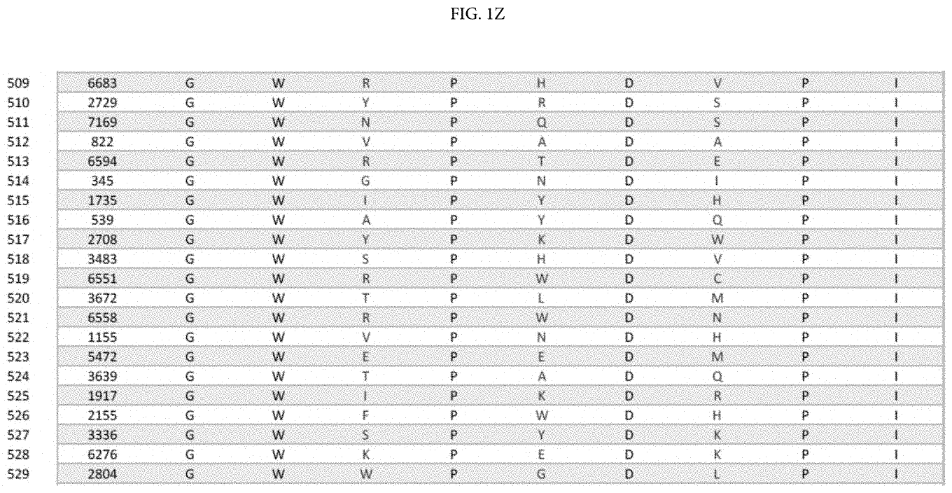

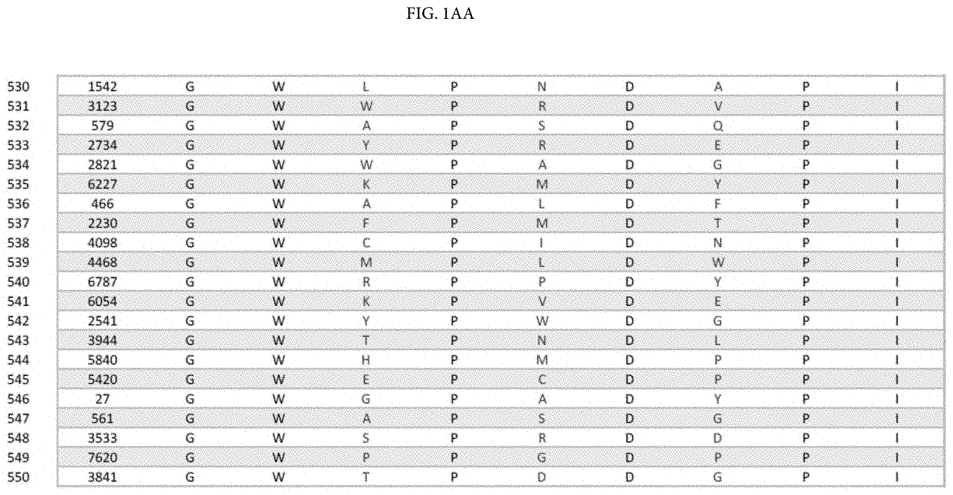

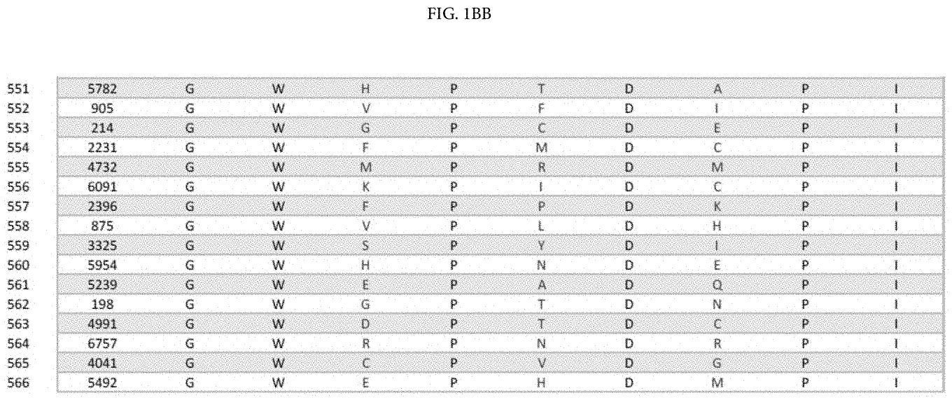

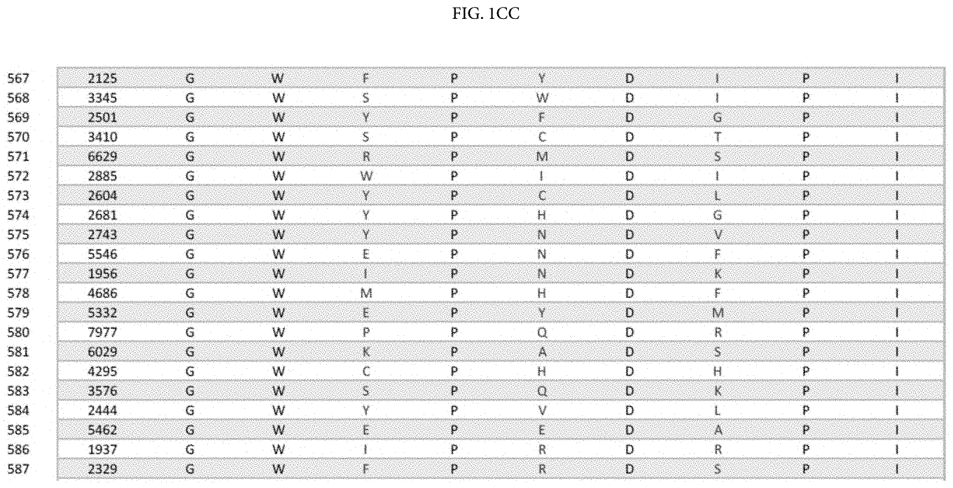

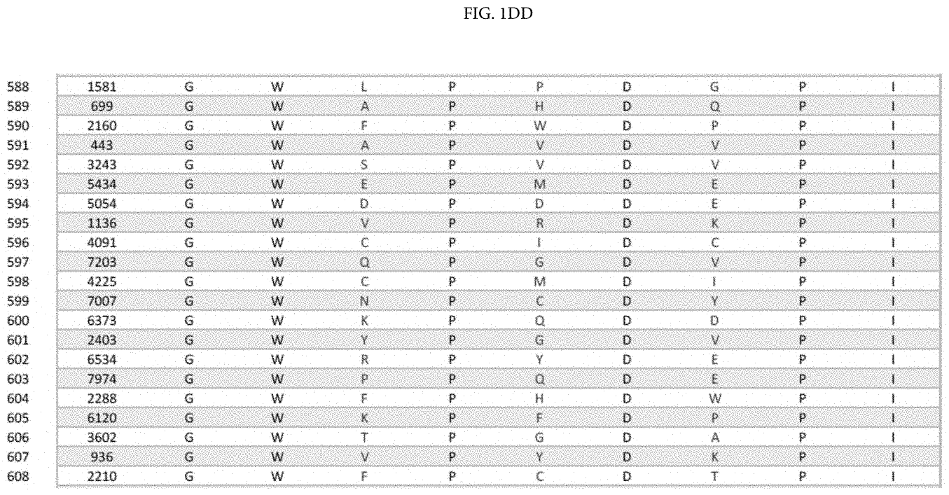

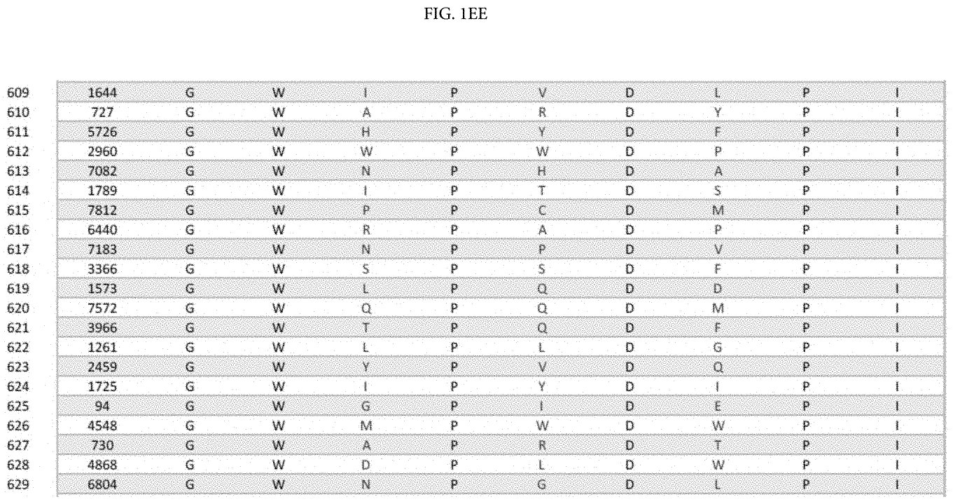

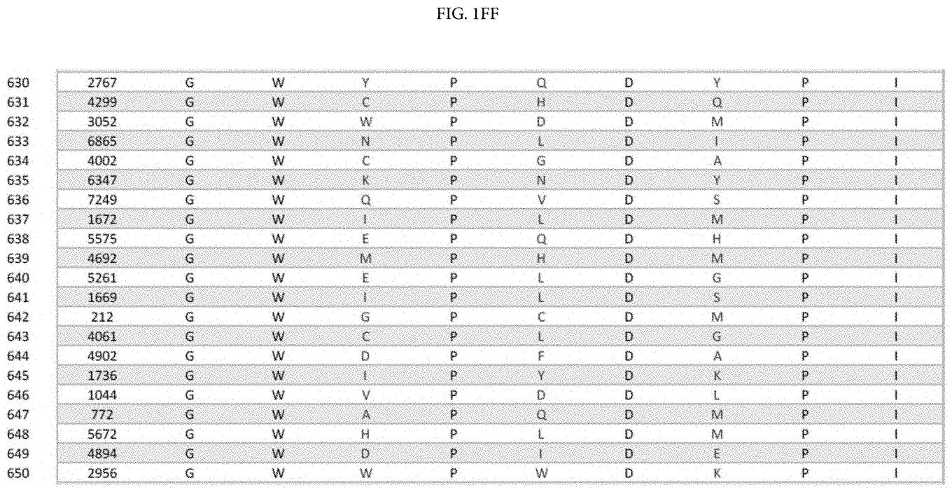

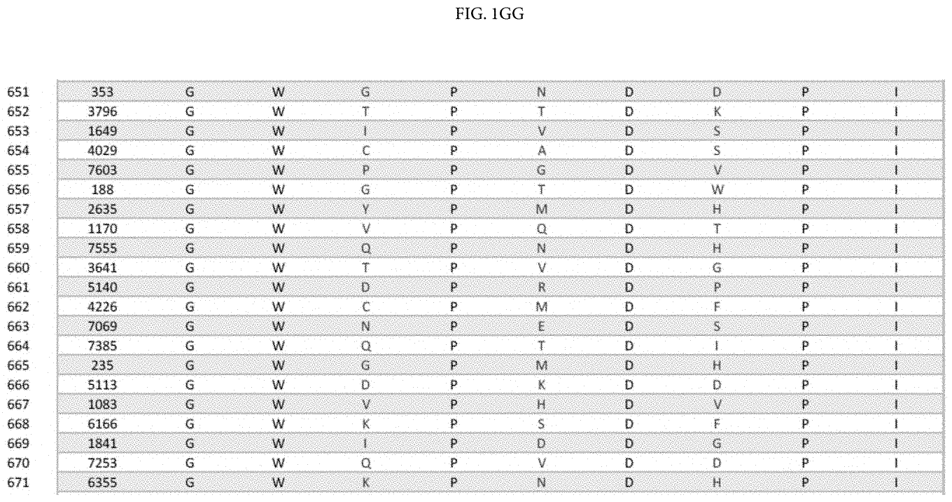

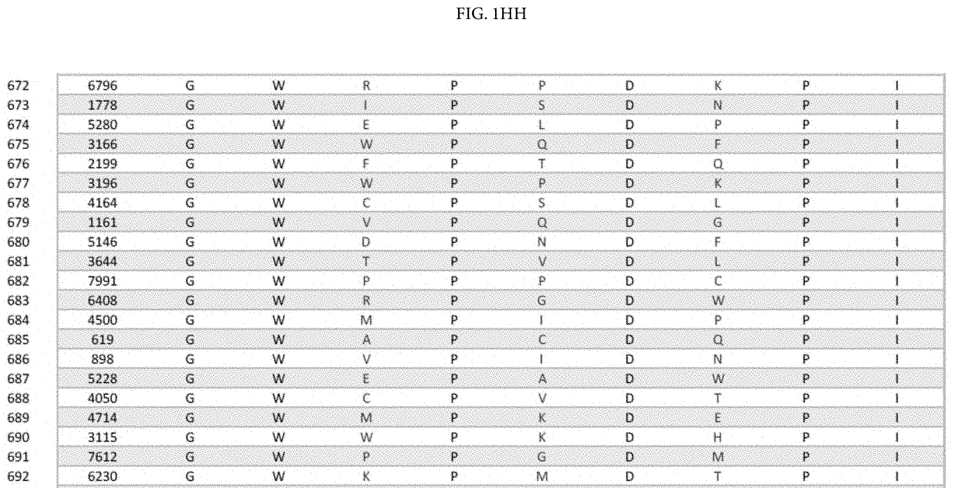

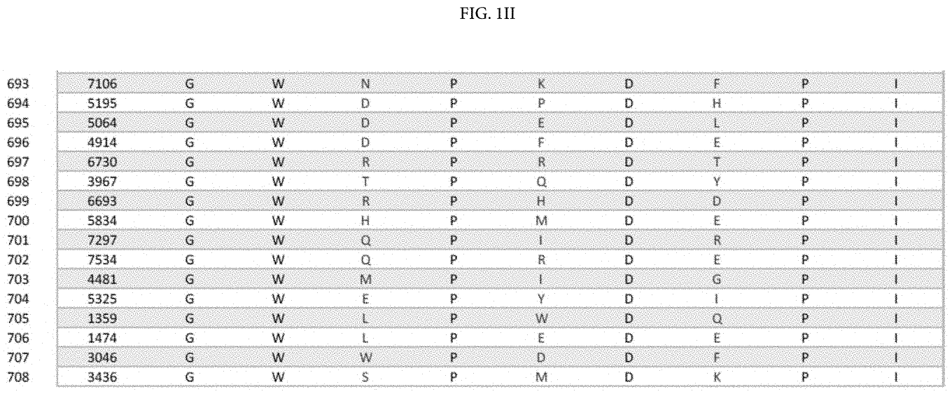

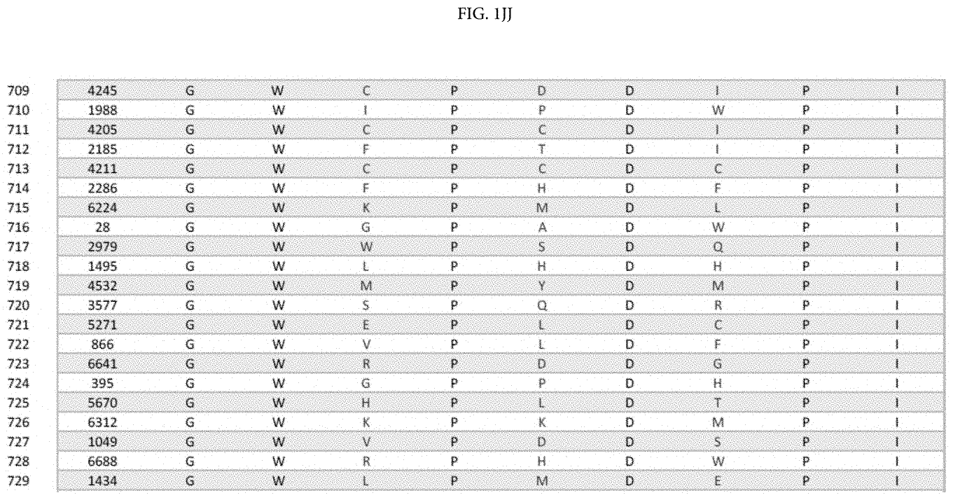

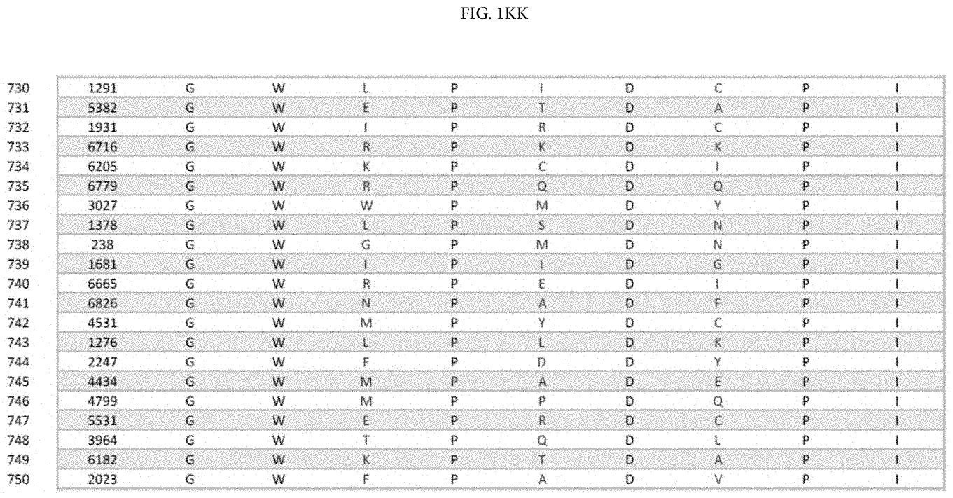

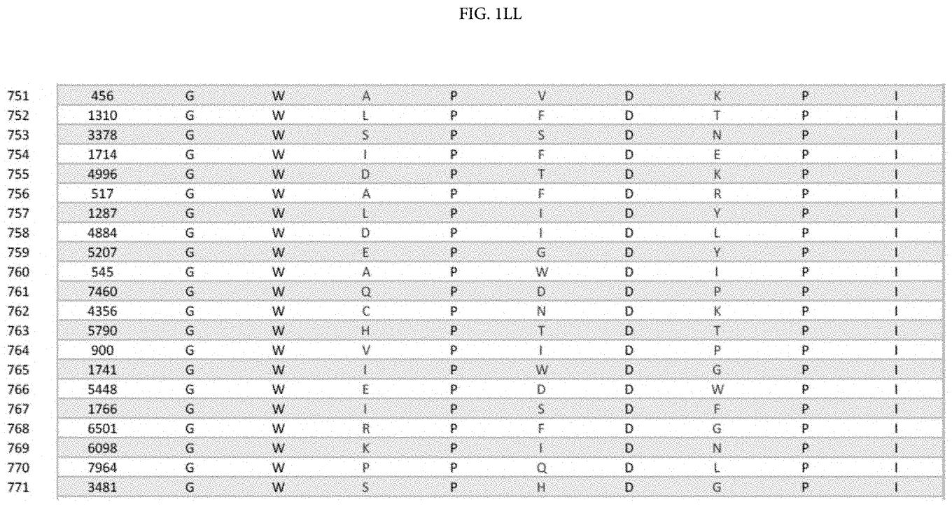

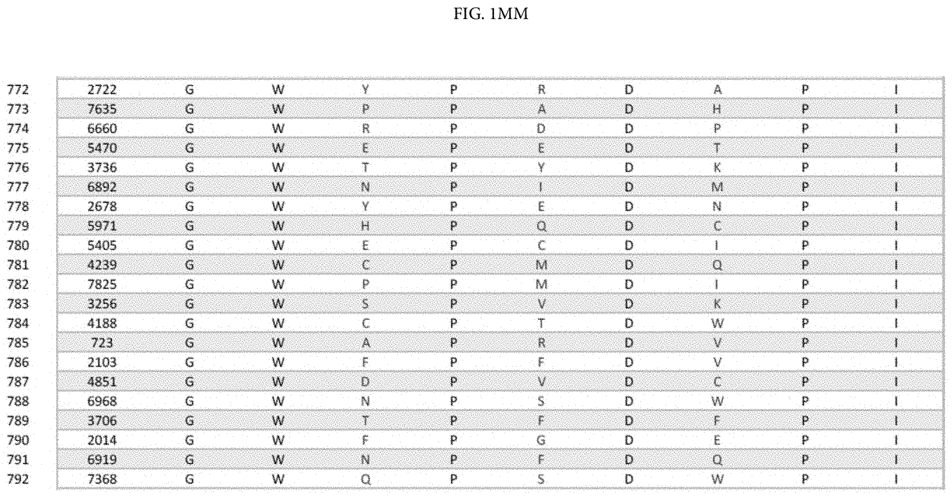

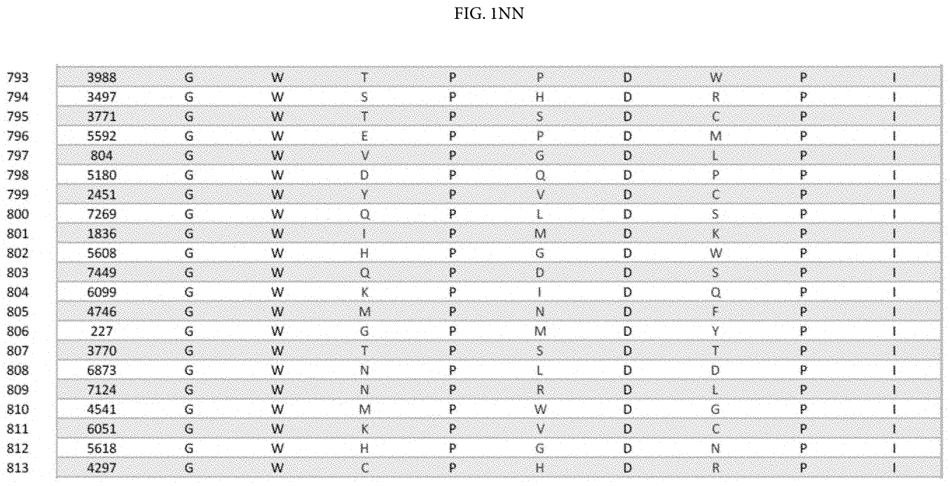

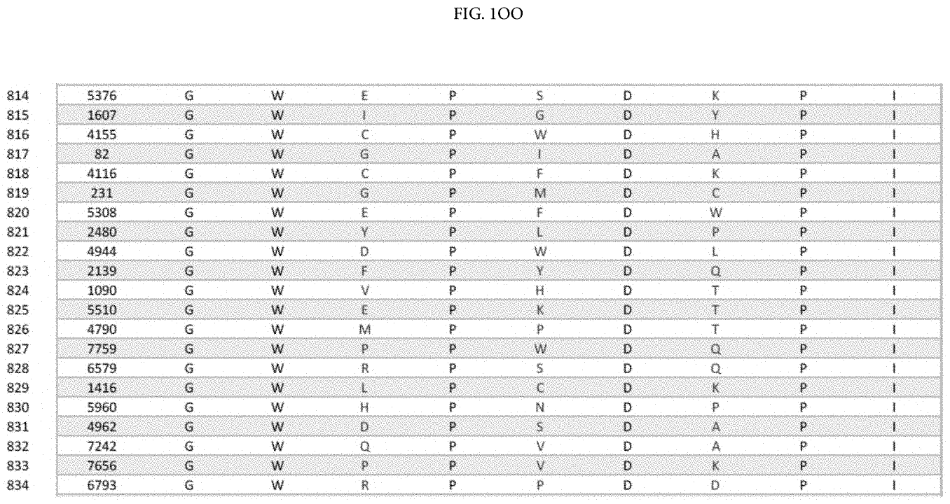

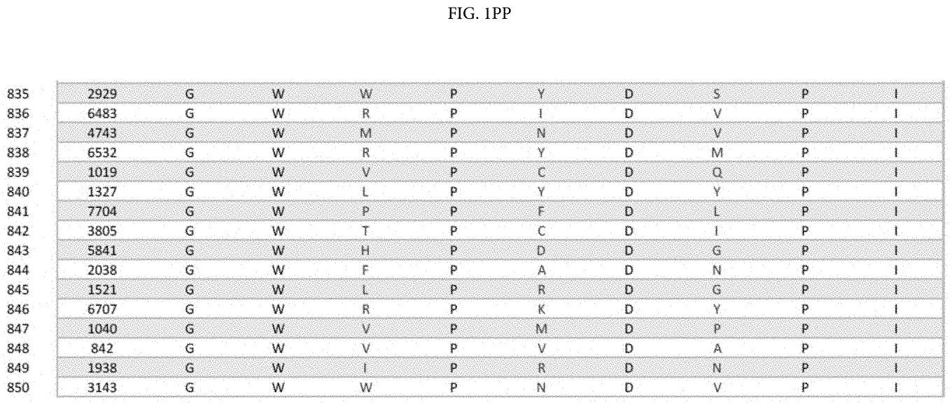

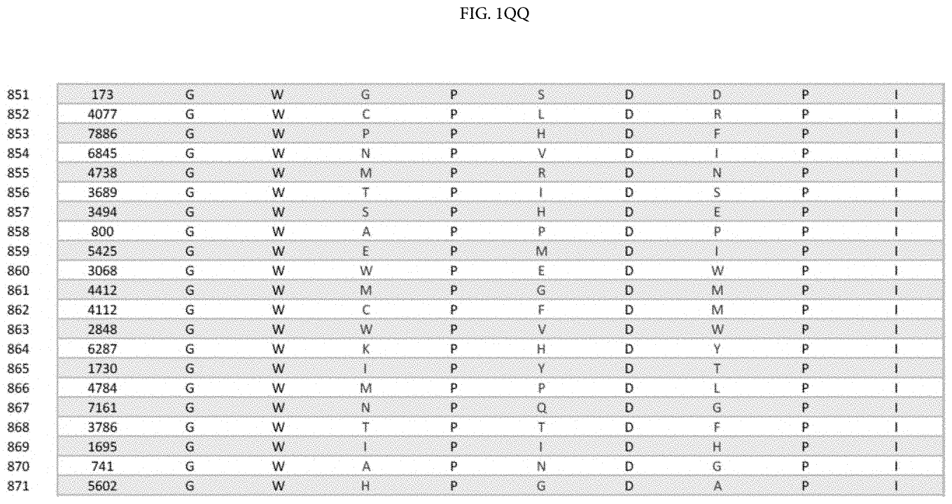

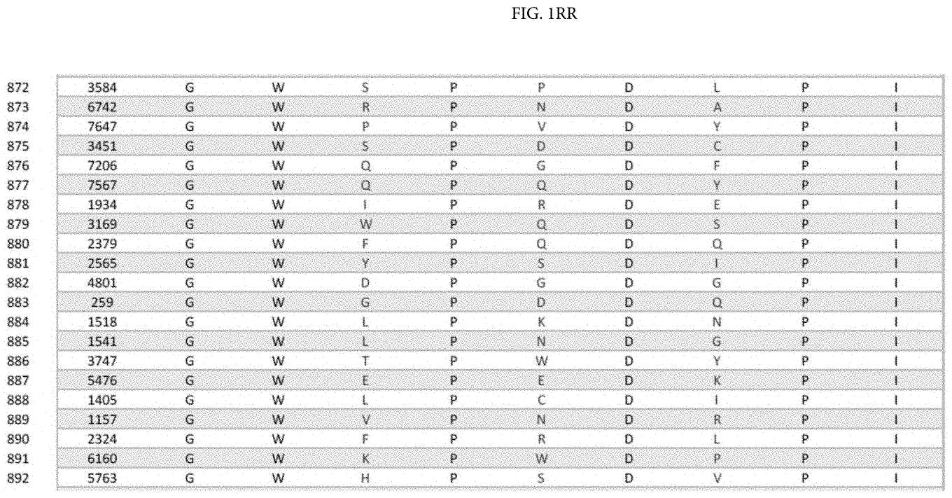

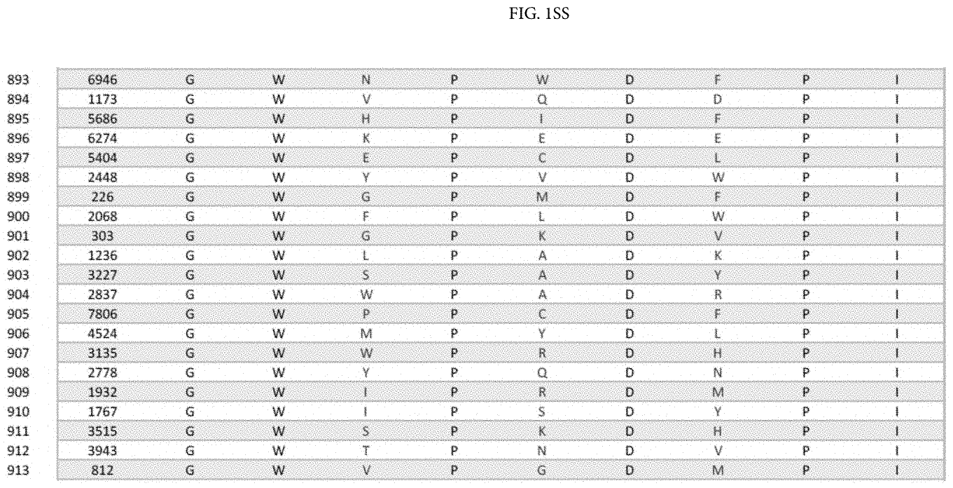

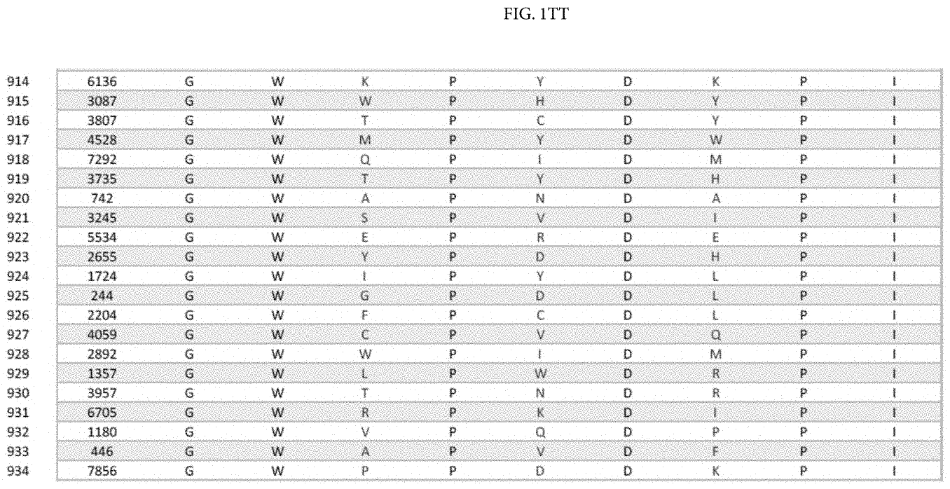

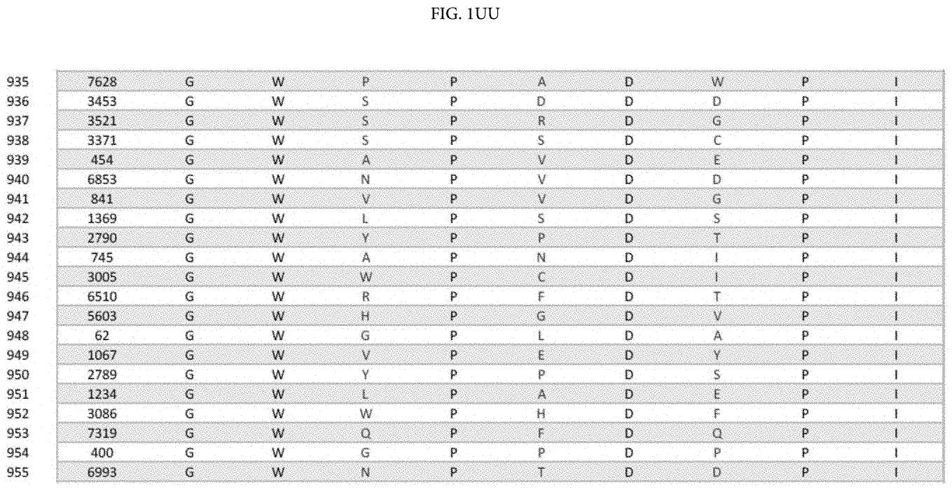

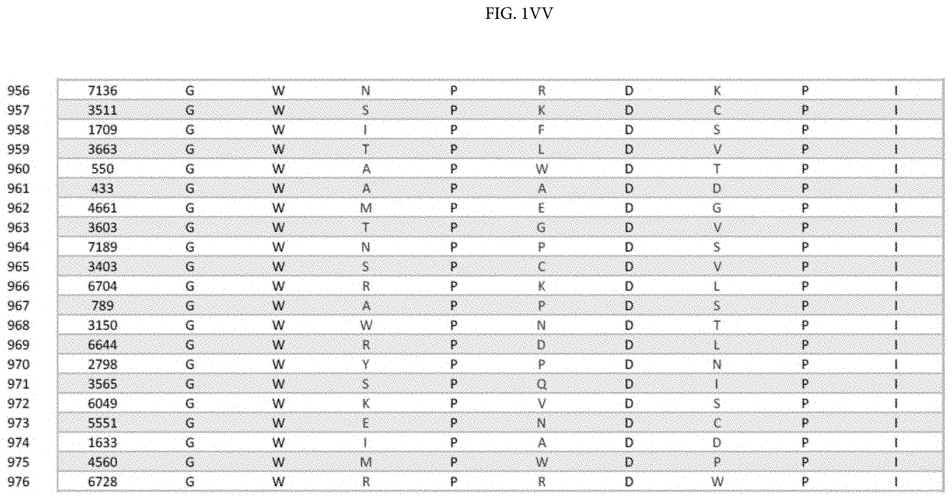

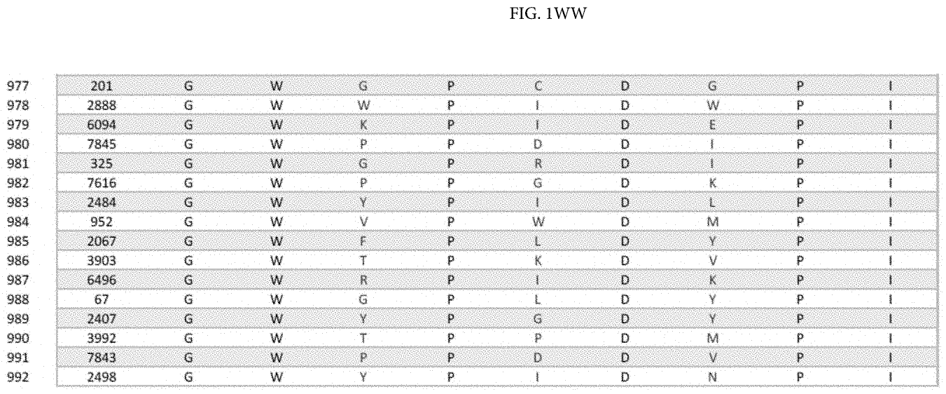

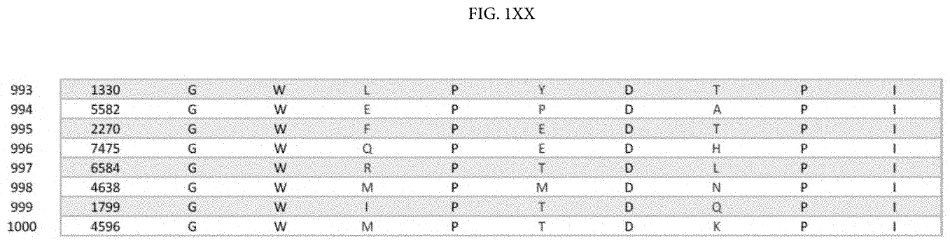

Thus, the DNA fragments corresponding to the wild type and variant epitopes, respectively, were amplified by PCR and cloned into pG8SAET phagemid vector that allows the expression of epitopes at high copy numbers as peptides fused to phage cpVlll. The amino acids at the MHC-binding anchor positions were maintained within the epitope, while mutations were introduced at positions responsible for interaction with TCR. As each variant epitope has random amino acid substitutions (mutations) at 3 defined positions within the wild type epitope, the theoretical complexity of the library is 8×10³ individual members. FIGS. 1A-1XX is a table displaying 1000 randomly selected peptides from said library. The phage library has a complexity of 10,500 original clones.

### Cell Proliferation Assays

PBLs are obtained from an individual of interest having said cancer as well as from a healthy individual (or population of healthy individuals). The Individual peptides are then assayed for its interaction with PBMCs from said individual and from a healthy individual (or population of healthy individuals) based on an in vitro proliferation assay.

### In Vitro Stimulation:

The PBMCs are stimulated by culturing in a 96-well flat-bottom plate (2.5×10⁵ cells/well) with 10⁷-10¹⁰ phage particles/well corresponding to particular epitope variant for 72 hours at 37° C. in CO₂ incubator. The gating strategy involves exclusion of doublets and dead cells; 10,000 lymphocytes (R1) are gated for a CD4+ versus CD8+ dot-plot graph to measure CD4+ IFN-y+, CD8+ IFN-y+ and proliferation percentages of CD4+CD8− and CD4−CD8+ cells.

Total cell proliferation and CD4+ and CD8+ T-cell responses are evaluated by using intracellular staining (ICS) for IFN-y both ex vivo and in vitro by stimulating fresh lymphocytes for 6 hours or 72 hours, respectively. During the last 4 hours, 1 μl/well Monensin (2 uM) (a protein transport inhibitor) is added to the culture. The cells are stained with fluorescence-labeled monoclonal antibodies against CD4 and CD8 for 30 minutes at room temperature, are fixed with fixation buffer and, after washing, the cells are permeabilized with permeabilization wash buffer, and then are labeled for 30 minutes with anti-IFN-y antibody in the dark. The cells are analyzed on FACSCalibur Cytometer using CellQuest software data acquisition and analysis program from BD Bioscience and operates in the Macintosh environment on the FACSCalibur cytometers; at least 10,000 events are collected.

### Selection of Peptides for Treatment of the Cancer Afflicting Said Individual

(a) Group I—peptides which induce proliferation of PBMCs of said afflicted individual and in said healthy population

(b) Group II—peptides which induce proliferation of PBMCs of said afflicted individual but not in said healthy population

(c) Group III—peptides which do not induce proliferation of PBMCs of said afflicted individual but induce proliferation in said healthy population

### Immunization of Individual with Positive Immunostimulatory Peptide

Peptides from one or more of Groups I, II and/or III are used as an inoculum to administer to said individual for treatment of the cancer afflicting said individual.

Epitopes/mimotopes bearing “absolute immunogenicity” will be the first vaccine component candidates. The “absolute immunogenicity” is defined as the set of peptides showing the highest capacity to induce the proliferation of PBMCs obtained from patient. The second vaccine component candidates are defined as the set of peptides showing decreased level of cell proliferation compared to cells from healthy subjects. Similarly, the third vaccine component candidates is defined as the set of peptides that show a similar immunogenicity using the cells both from healthy individuals and patients with cells.

FIGS. 1A-1XX shows the potency of these peptides as a component in a therapeutic agent or vaccine, are optionally determined using animal and preclinical models.

### Working Example II

Mimotope library PG5D A[G/F]PXXXXX[L/M], (SEQ ID NO: 34), (5X library) based on the mimotope AGPAAAAAL (SEQ ID NO: 35), was constructed as described in our NoeDominguez-Romero et al., (2014) Human Vaccines & Immunotherapeutics, 10(11):3201-3213, having a theoretical complexity of 3.2×10⁶ individual members (prepared at GenScript Corporation (Piscataway, N.J., USA). The mimotope variants comprising the combinatorial peptides, were generated using degenerate oligonucleotides encoding a library of mimotope variants with structural composition A[G/F]PXXXXX[L/M] (SEQ ID NO: 35), where X is any of 20 natural amino acids.

### Working Example 3

The proto-oncogene HER2, a 185-kDa membrane receptor type tyrosine kinase with 1255 amino acids is often overexpressed in a variety of human cancers such as breast, ovarian, lung and gastric cancers with limited expression in normal tissues Okugawa et al. (2000) Eur J Immunol. 30(11):3338-46. Okugawa et al. identified a human HER2 derived monomer peptide (HER2p63) (TYLPTNASL) (SEQ ID NO: 36), that can induce HER2-specific CTL in HLA-A2402-positive individuals. In the mouse Her2-derived epitope (TYLPANASL) (SEQ ID NO: 37), there is an Alanine instead of a Threonine at position #5, as in the human analog.

VELs were generated using the recombinant M13 phage display system based on a Her2-derived CTL epitope. The epitope variants comprising the combinatorial VELs, were generated using degenerate oligonucleotides encoding a library of epitope variants with structural composition TYXPXNXSL, (SEQ ID NO: 38), where X at positions 3, 5, and 7 is any one of the 20 natural amino acids. Ninety one variant epitopes were randomly selected from the VEL generated based on HER2-derived CTL epitope. The PBMC cells from a patient afflicted with breast cancer were cultured for 72 hours with 10⁷ phage/well as described above.

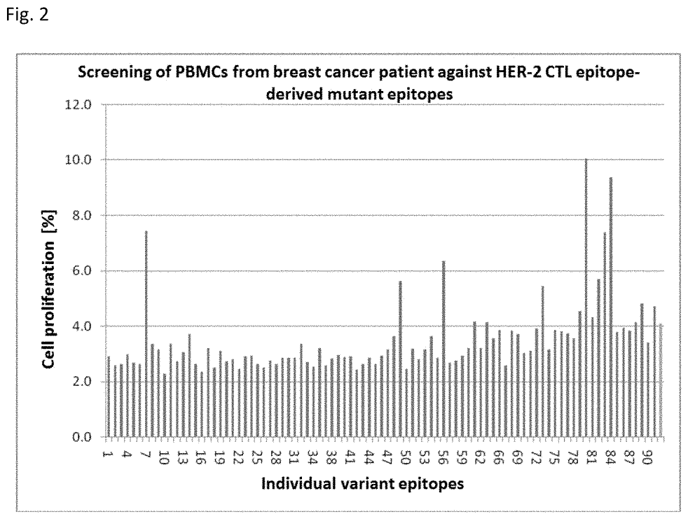

FIG. 2 displays the results of PBMC cell proliferation from a patient afflicted with breast cancer against a panel of HER2 CTL epitope-derived VEL library mutant/variant epitopes. The non-related phage always was resulting low level background proliferation as the majority of variant epitopes in this FIG. 2 (data not shown). As shown, there are about 10 variant clones resulting better immune stimulators than WT epitope expressing phage clone (the last clone in the FIG. 2).

1. A method of identifying a set of peptides for treatment against a disease or condition afflicting an individual, wherein the set of peptides comprises one or more peptides comprising (i) a T cell epitope of an antigen expressed in said individual and/or (ii) variants of said T-cell epitope, comprising:
(a) generating a combinatorial variable epitope library (VEL) wherein said VEL comprises a plurality of peptides, each said peptide comprising a T cell epitope or variant thereof, wherein the length of each said T cell epitope or variant thereof, ranges from 8 to 11 amino acids, wherein the amino acid residues at MHC class I-anchor positions of said T cell epitope and its variant are identical, wherein the sequence of said T cell epitope and said variant thereof differ in at least two residues,
(b)
(i) incubating said T cell epitope or a variant thereof, with peripheral blood mononuclear cells (PBMCs) from a healthy individual (or a population of healthy individuals) under conditions suitable for inducing proliferation of PBMCs;
(ii) incubating said T cell epitope or variant thereof, with PBMCs from said individual afflicted with said disease or condition under conditions suitable for inducing proliferation of PBMCs, wherein said afflicted individual has a MHC Class I haplotype which is similar to the MHC Class I haplotype of said healthy individual,
(iii) comparing the proliferation of said T cell epitope and of each said variant thereof, in step (b)(i) versus step (b)(ii), thereby identifying four peptide groups:
(a) Group I—peptides which induce proliferation of PBMCs of said afflicted individual and in said healthy population
(b) Group II—peptides which induce proliferation of PBMCs of said afflicted individual but not in said healthy population
(c) Group III—peptides which do not induce proliferation of PBMCs of said afflicted individual but induce proliferation in said healthy population
wherein each said peptide Group, or a combination of two or more of Groups I, II, III and/or IV, identifies a set of peptides for treatment against said disease or condition afflicting said individual.

2. The method of claim 1, wherein said method comprises chemical synthesis of said peptides.

3. The method of claim 1, wherein the chemical synthesis is performed in the wells of a 96 well plate.

4. The method of claim 1, wherein when the sequence of said T cell epitope and its variant thereof differ at only two amino acid residues, the VEL comprises at least 100 variant peptides.

5. The method of claim 1, wherein when the sequence of said T cell epitope and its variant thereof differ at only three amino acid residues, the VEL comprises at least 1000 variant peptides.

6. The method of claim 4 wherein said variants are selected randomly.

7. The method of claim 5, wherein said variants are selected randomly.

8. The method of claim 1, wherein the sequence of said CTL epitope is GWEPDDNPI.

9. The method of claim 8, wherein the derivatives of peptide epitope GWEPDDNPI is GWXPXDXPI, wherein “X” is any of 20 amino acids.

10. A method of identifying a set of peptides for treatment against a disease or condition afflicting an individual, wherein the set of peptides comprises one or more peptides (i) a mimotope of a T cell epitope of antigen expressed in said patient and/or (ii) variants of said T cell mimotope, comprising:
(a) generating a combinatorial variable epitope library (VEL) wherein said VEL comprises a plurality of peptides, each said peptide comprising a T cell mimotope or variant thereof, wherein the length of each said T cell mimotope or variant thereof ranges from 8 to 11 amino acids, wherein the amino acid residues at MHC class I-anchor positions of said T cell mimotope and its variant thereof are identical, wherein the sequence of said T cell mimotope and said variant thereof differ in at least two residues,
(b)
(i) incubating said T cell mimotope or variant thereof, with peripheral blood mononuclear cells (PBMCs) from a healthy individual (or population of healthy individuals) under conditions suitable for proliferation of PBMCs,
(ii) incubating said T cell mimotope or variant thereof, with PBMCs from said individual afflicted with said disease or condition under conditions suitable for proliferation of PBMCs, wherein said afflicted individual has MHC Class I haplotype which is similar to the MHC Class I haplotype of said heathy individual (or population of healthy individuals);
(iii) comparing the proliferation of said T cell mimotope and of each said variant thereof, in step (b)(i) versus step (b)(ii), thereby identifying four peptide groups:
(a) Group I—peptides which induce proliferation of PBMCs of said afflicted individual and in said healthy population
(b) Group II—peptides which induce proliferation of PBMCs of said afflicted individual but not in said healthy population
(c) Group III—peptides which do not induce proliferation of PBMCs of said afflicted individual but induce proliferation in said healthy population
wherein each said peptide Group, or combination of two or more of Groups I, II, III and/or IV, identifies a set of peptides for treatment against said disease or condition afflicting said individual.

11. The method of claim 10, wherein said method comprises chemical synthesis of said peptides.

12. The method of claim 10, wherein the chemical synthesis is performed in the wells of a 96 well plate.

13. The method of claim 10, wherein when the sequence of said T cell mimotope and said variant thereof differ at only two amino acid residues, the VEL comprises at least 100 variant peptides.

14. The method of claim 10, wherein when the sequence of said T cell mimotope and said variant thereof differ at only three amino acid residues, the VEL comprises at least 1000 variant peptides.

15. The method of claim 13 wherein said variants are selected randomly.

16. The method of claim 14, wherein said variants are selected randomly.

17. The method of claim 10, wherein the amino acid sequence of said CTL mimotope is AGPAAAAAL.

18. The method of claim 17, wherein a variant of said CTL epitope mimotope AGPAAAAL is selected from the group consisting of A[G/F]PXXXXX[L/M], where “X” is any of 20 amino acids and AGPXAXAXL, where “X” is any of 20 amino acids.

19. The method of claim 1, further comprising immunization of the afflicted individual with a formulation comprising at least one or with the mixture of up to 100 variant peptides identified in step (b) and pharmaceutically acceptable carrier.

20. The method of claim 10, further comprising immunization of the afflicted individual with a formulation comprising at least one or with the mixture of up to 100 variant peptides identified in step (b) and pharmaceutically acceptable carrier.

21. The method of claim 1, wherein the sets of peptide epitopes of said combinatorial variable epitope library (VEL) are expressed by one or more of the group consisting of plasmid DNA, a viral vector and a microorganism.

22. The method of claim 21, wherein the sets of peptide epitopes of said combinatorial variable epitope library (VEL) are present at the surface of said microorganism, wherein said microorganism is selected from the group consisting of bacteriophage, yeast and bacteria.

23. The method of claim 1, wherein the sets of peptide epitopes of said combinatorial variable epitope library (VEL), are expressed on the surface of insect cells in combination with a MHC class I molecule.

24. The method of claim 10, wherein the sets of peptide mimotopes of said combinatorial variable epitope library (VEL), are expressed by one or more of the group consisting of plasmid DNA, a viral vector and a microorganism.

25. The method of claim 26, wherein the sets of peptide mimotopes of said combinatorial variable epitope library (VEL) are present at the surface of such a microorganism, wherein said microorganism is selected from the group consisting of bacteriophage, yeast and bacteria.

26. The method of claim 10, wherein the sets of peptide mimotopes of said combinatorial variable epitope library (VEL) are expressed on the surface of insect cells in combination with a MHC class I molecule.

27. The method of claim 1, wherein said plurality of peptides comprises three or more peptides.

28. The method of claim 10, wherein said plurality of peptides comprises three or more peptides.
